(19) United States
(12) Patent Application Publication
(10) Pub. No.: US 2010/0297251 A1
(43) Pub. Date: Nov. 25, 2010

(54) ENCAPSULATED PARTICLES FOR ENTERIC RELEASE

(75) Inventors: Richard B. Timmons, Arlington, TX (US); Ceren Susut, Arlington, VA (US); Donald E. Owens, III, Austin, TX (US); J. Brian Windsor, Austin, TX (US); Rachel Kennedy Khorzad, Austin, TX (US)

Correspondence Address: CHALKER FLORES, LLP, 2711 LBJ FRWY, Suite 1036, DALLAS, TX, 75234, US

(73) Assignees: BOARD OF REGENTS, THE UNIVERSITY OF TEXAS SYSTEM, Austin, TX (US); AEONCLAD COATINGS, LLC, Austin, TX (US)

(21) Appl. No.: 12/785,271

(22) Filed: May 21, 2010

Related U.S. Application Data
(63) Continuation-in-part of application No. 10/931,480, filed on Sep. 1, 2004.

(51) Int. Cl. A61K 9/58; A61K 31/4196; A61K 31/343; A61K 31/12; A61K 38/13; A61K 31/56; A61K 31/19; A61K 31/55; A61K 31/52

(52) U.S. Cl. 424/501; 514/384; 514/462; 514/682; 514/20.5; 514/26; 514/173; 514/570; 514/217; 514/263.1; 427/2.14

(57) ABSTRACT

The present invention provides a system for delivery of an enteric coated active agent generally resistant to disintegration in an neutral environment having one or more active agents encapsulated by a polymer coating formed by chemical vapor deposition of one or more monomers on the one or more active agents to form a chemical vapor deposition polymer coating that controls the release of the one or more active agents in the gastrointestinal tract.

### CROSS-REFERENCE TO RELATED APPLICATIONS

This application is a continuation-in-part and claims priority based on U.S. patent application Ser. No. 10/931,480, filed Sep. 1, 2004, the contents of each of which are incorporated by reference herein in their entireties.

### TECHNICAL FIELD OF THE INVENTION

The present invention relates in general to the field of encapsulation of materials, and more particularly, to compositions and methods for encapsulation of an agent or particle for controlled release in the intestinal tract via a coating layer(s) using gas phase chemical vapor deposition.

### STATEMENT OF FEDERALLY FUNDED RESEARCH

None.

### INCORPORATION-BY-REFERENCE OF MATERIALS FILED ON COMPACT DISC

None.

### BACKGROUND OF THE INVENTION

Without limiting the scope of the invention, its background is described in connection with encapsulation of particles or agents.

Encapsulation, in which an agent, such as an active pharmaceutical ingredient (API) or drug, is surrounded or coated by at least one layer of a surface, has many beneficial uses. Unfortunately, current methods of encapsulation generally require a number of technical steps and result in encapsulated products with poor stability. In addition, most methods result in low product yields, due, in part, to the limited tolerance of the starting materials to industrial operating conditions and the numerous technical difficulties associated with the encapsulation process, with product recovery and inadequate recycling from the reaction systems.

Enteric drugs are one example of a current use for encapsulation technology. Especially challenging is the dissolution and controlled delivery of poorly soluble agents, such as APIs. A common technical challenge encountered with traditional enteric coatings is that a large percentage of material is needed to create the enteric coating, ranging from 20 to upwards of 90% of the total mass of a drug formulation. In some instances, the enteric tablet or dosage forms become so large they are not easily administered in a single dose and must be taken multiple times a day or completely reformulated. The present invention overcomes this difficulty by applying the coating layer at an individual particle level wherein very small quantities of coating (<1%) can impart enteric behavior. For APIs, this is extremely beneficial because excipient loading is decreased and drug potency is increased. With this technology dosage form size and frequency of use can be decreased because such a small amount of coating is required and hence the potency of the coated material is much higher.

Furthermore, because standard enteric coatings require such a high loading of excipient to achieve enteric behavior, the resulting increased diffusional distance that an agent must travel can result in incomplete or inefficient drug release. This delay in release of encapsulated material can lead to suboptimal delivery and hence lower bioavailability. One of the advantages of this invention is the ability to individually coat particles on both the micro and nano scales, which greatly increases bioavailability.

Current approaches to particle encapsulation include layer-by-layer assembly of polyelectrolytes, emulsion-solvent evaporation processes, formation of hydrogel films, and the preparation of systems based on thiolated polymers, sol-gel carriers, and granulation techniques. While current approaches do provide satisfactory results for introducing particles to an environment; these approaches are complex, involve a number of technical steps, generate large amounts of waste products, are often inadequate in truly controlling the introduction of the particle into the environment, and these processes are not able to apply coating layers at the particle level. The present invention overcomes the complexity and generation of waste by utilizing a solvent free chemical vapor deposition process.

Clearly, there remains a need to provide for more efficient compositions, systems and methods for encapsulation of a particle or agent within a controlled release coating layer that is targeted for release or delivery in the intestinal tract.

### SUMMARY OF THE INVENTION

The present invention solves the current problem associated with inefficient systems and methods for encapsulation of agents for delivery to the intestinal tract. The present invention provides for a novel method of encapsulating an agent, such as an API, within a controlled release coating layer(s) using a gas phase chemical vapor deposition process.

Generally, and in one form, the present invention provides for encapsulation of individual agents, such as an API or API/excipient mixture, within a coating layer(s) via a gas phase chemical vapor deposition process, wherein said coating layers are applied using continuous or pulsed plasma enhanced chemical vapor deposition processes. Individual particles are coated with at least one layer of a coating material. The coating layer(s) can be applied in sequential steps to create a gradient layer using variable duty cycles, monomer flow rates, peak power, reaction times, loading, reaction pressures, or various combinations thereof.

In another form, the present invention provides for the encapsulation of individual agents, such as an API or API/excipient mixture, that may exhibit enhanced solubility, or alternatively, achieve supersaturation; using continuous or pulsed plasma enhanced chemical vapor deposition processes. Individual particles are coated with at least one layer of a coating material. The coating layer(s) can be applied in sequential steps to create a gradient layer using variable duty cycles, monomer flow rates, peak power, reaction times, loading, reaction pressures, or various combinations thereof.

The present invention is particularly beneficial because the coating is applied at the drug particle level rather than the tablet level allowing faster dissolution upon entering the upper small intestine. This in turn leads to increased bioavailability because more encapsulated material is released within the high absorption region of the upper small intestine.

In addition, the coating material influences particle release into an environment. The coating material and, hence, targeted delivery of the agent to the intestinal tract, is dependent on the encapsulation process as well as the composition of the coating of the present invention. In one embodiment, the coating layer(s) is comprised of enteric compounds that are generally resistant to disintegration in acidic media but will begin to disintegrate in solutions with pH greater than 4.5. Thus, the encapsulated particle remains intact within the acidic environment of the stomach, but rapidly disintegrates and allows for the dissolution and delivery of the core agent, such as an API or API/excipient mixture, to the intestinal tract for absorption.

In another embodiment, the coating layers(s) are comprised of pH responsive polymers that switch from uncharged in acidic media (stomach) to charged in neutral media (intestines) and in doing so become unstable and begin swelling and/or disintegrating. Thus, the encapsulated particle remains intact within the acidic environment of the stomach, but rapidly disintegrates and/or swells and allows for the dissolution and delivery of the core agent, such as an API or API/excipient mixture, to the intestinal tract for absorption.

This encapsulation process is solvent free, which allows highly soluble as well as highly insoluble drug particles to be easily coated in dry form. This invention overcomes the difficulties of using standard wet chemistry techniques with aqueous solutions wherein highly soluble particles dissolve before they can be coated. Likewise the use of organic and sometimes toxic solvents and plasticizers to apply a coating is not required and hence the chance of incorporation of these undesirable compounds is eliminated.

The present invention also provides for a system for delivery of an agent for controlled release in the intestinal tract, comprising a particle or agent core encapsulated by one or more coating layers applied directly to the surface of the core via a gas phase chemical vapor deposition process.

Reaction conditions that promote polymerization and/or encapsulation generally include power input, peak power, coating time, duty cycle, flow rate of the monomer, reactor pressure, and quantity of the particles. By altering one or more of the reaction conditions, encapsulation is controlled. By controlling encapsulation, one can control the release behavior and rate of release of the encapsulated constituents into an environment. The coating layers are polymeric materials of varying cross-linked density, functionality, hydrophilicity, hydrophobicity, molecular weight and thickness. Aspects of the coating that can be controlled include film growth, thickness, number, density and quality of one or more monomeric functional groups, hydrophilicity or hydrophobicity, wettability, linearity, cross-linking, molecular weight, and various combinations thereof.

In another form, the present invention is a method for the encapsulation of an agent, such as individual API or API/excipient composite particles, within a controlled release coating layer(s) via a gas phase chemical vapor deposition process, wherein said coating layers are applied using continuous or pulsed plasma enhanced chemical vapor deposition processes.

In still another form, the present invention provides methods and systems for controlling release of one or more particles into an environment. Drug release profiles can be customized by uniquely tailoring coatings to allow for modified or sustained release at the micro or nanoparticle level. In addition, the technology can also be used to functionalize particles for targeted delivery.

The present invention provides a system for delivery of an enteric coated active agent generally resistant to disintegration in an neutral environment having one or more active agents encapsulated by a polymer coating formed by chemical vapor deposition of one or more monomers on the one or more active agents to form a chemical vapor deposition polymer coating that controls the release of the one or more active agents in the gastrointestinal tract.

The present invention also includes a method of encapsulating one or more active agents in a chemical vapor deposition layer for controlled release in the intestinal tract by providing one or more active agents in a reaction chamber; adding one or more monomers to the reaction chamber; forming a plasma of the one or more monomers to make a chemical vapor; depositing the chemical vapor on the one or more active agents to encapsulate the one or more active agents in a chemical vapor deposition polymer coating for controlled release in the intestinal tract.

The present invention includes a method for delivering one or more active agents to achieve supersaturation in the gastrointestinal tract by providing a pharmaceutical composition having a chemical vapor deposition layer to control the release to a subject, wherein the pharmaceutical composition comprises one or more active agents encapsulated with a polymer coating that has been deposited by chemical vapor deposition for controlled release at a pH 4.5 or higher in the intestinal tract.

An enteric coated pharmaceutical composition having a chemical vapor deposition layer is also provided and includes one or more active agents encapsulated by a polymer coating formed by chemical vapor deposition on the one or more active agents to form a chemical vapor deposition polymer coating that controls the release of the one or more active agents at a pH 4.5 or higher.

In yet another form the present invention provides for compositions prepared by systems and methods of the present invention. Compositions include organic and inorganic compositions; such as pharmaceutical compositions, for example.

Those skilled in the art will further appreciate the above-noted features and advantages of the invention together with other important aspects thereof upon reading the detailed descriptions that follows in conjunction with the drawings.

### BRIEF DESCRIPTION OF THE DRAWINGS

For more complete understanding of the features and advantages of the present invention, reference is now made to the detailed description of the invention along with the accompanying figures, wherein:

FIG. 1 depicts dissolution testing results for uncoated amorphous Itraconazole (ITZ) particles prepared via a hot melt extrusion process (n=3).

FIG. 2 depicts dissolution testing results for poly(methacrylic acid)-co-poly(methyl methacrylate) (PMAA-co-PMMA) coated amorphous ITZ particles prepared via a hot melt extrusion process (n=3).

FIG. 3 depicts dissolution testing results for perfluorohexane (C₆F₁₄) coated amorphous ITZ particles prepared via a hot melt extrusion process (n=3).

FIG. 4 depicts dissolution testing results for C₆F₁₄ coated amorphous ITZ particles prepared via a hot melt extrusion process with sodium dodecyl sulfate (SDS) surfactant added to the dissolution media (n=3).

FIG. 5 depicts dissolution testing results for PMAA-co-PMMA coated crystalline ITZ particles with SDS surfactant added to the dissolution media (n=3).

FIG. 6 depicts dissolution testing results for C₆F₁₄ coated crystalline ITZ and Eudragit L100 blended drug particles with SDS surfactant added to the dissolution media (n=3).

FIG. 7 depicts in-vivo animal model testing results for PMAA-co-PMMA coated amorphous ITZ particles prepared via a hot melt extrusion process (n=3).

FIG. 8 depicts in-vivo animal model testing results for C₆F₁₄ coated amorphous ITZ particles prepared via a hot melt extrusion process (n=3).

FIG. 9 depicts dissolution testing results for PMAA-co-PMMA coated amorphous ITZ antisolvent precipitation particles (n=3).

FIG. 10 depicts dissolution testing results for C₆F₁₄ coated amorphous ITZ antisolvent precipitation particles (n=3).

FIG. 11 depicts dissolution testing results for C₆F₁₄ coated amorphous ITZ antisolvent precipitation particles with SDS surfactant added to the dissolution media (n=3).

FIG. 12 depicts a SEM image showing the surface morphology of uncoated amorphous ITZ particles at a magnification of 7910×. Image was taken using a Leo 1530 scanning electron microscope.

FIG. 13 depicts a SEM image showing the surface morphology of C₆F₁₄ coated amorphous ITZ particles at a magnification of 3810×. Image was taken using a Leo 1530 scanning electron microscope.

FIG. 14 depicts a SEM image showing the surface morphology of PMAA-co-PMMA coated amorphous ITZ particles at a magnification of 3770×. Image was taken using a Leo 1530 scanning electron microscope.

FIG. 15 depicts Fourier transform infrared spectroscopy analysis of the PMAA-co-PMMA coating deposited on a potassium bromide (KBr) powder using the plasma enhanced chemical vapor deposition technique.

FIG. 16 depicts Fourier transform infrared spectroscopy analysis of the C₆F₁₄ coating deposited on a KBr powder using the plasma enhanced chemical vapor deposition technique.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific aspects and embodiments discussed herein are merely illustrative of specific ways to make and use the invention and do not limit the scope of the invention.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects. The terminology herein is used to describe specific embodiments of the invention, but their usage does not delimit the invention, except as outlined in the claims.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

In the description which follows like parts may be marked throughout the specification and drawing with the same reference numerals, respectively. The drawing figures are not necessarily to scale and certain features may be shown exaggerated in scale or in somewhat generalized or schematic form in the interest of clarity and conciseness.

Discovering new and improved techniques for encapsulation of an agent has become one of today's fastest growing areas of research. While many of these techniques have biologic, chemical, and pharmaceutical applications, other applicable fields include electronics, the food industry, optics, data management, agriculture, and material sciences, as examples. In general, the primary purpose of encapsulation is to be able to control, target, delay and/or increase the concentration, timing and rate of release of an agent into a specific environment.

Significantly, poor drug solubility can be overcome with a uniquely tailored coating to allow for modified or sustained release in a specific environment. In the pharmaceutical and medical device industry, another purpose for particle encapsulation is to improve agent effectiveness when introduced into a biologic system and to reduce any negative consequences associated with introduction of the agent. The ability to apply coating layers to an agent, such as an API, at the individual particle level greatly reduces the agglomeration of drug particles and increases the bioavailability of the agent. In addition, the encapsulated constituents should reduce costs associated with its introduction, e.g., reduce dosing, reduce administration of concomitant agents or particles, and reduce the necessity for specialized personnel and/or equipment. The present invention is capable of accomplishing these and other tasks as is further described below.

Controlled release in the present invention is the introduction of a particle into an environment, such as the intestinal tract, wherein the manipulated degradation or dissolution of a coating layer(s) surrounding a core comprising an agent allows for control of the release of said agent into that environment in a way that would otherwise not be obtainable without the addition of said coating layer(s).

The present invention provides a system for delivery of an enteric coated active agent generally resistant to disintegration in an neutral environment having one or more active agents encapsulated by a polymer coating formed by chemical vapor deposition of one or more monomers on the one or more active agents to form a chemical vapor deposition polymer coating that controls the release of the one or more active agents in the gastrointestinal tract. The active agents include analgesic agents, anti-inflammatory agents, anti-infective agents, proteinaceous agents, enzymatic agents, or a combination thereof. In one embodiment, the active agents include Itraconazole, aspirin, Ketoprofen, Albuterol sulfate, cabamazepine, cyclosporin A (CsA), Danazol, ketoconazole, Itraconazole, voriconazole, Naproxen, Repaglinide, Tacrolimus, bovine insulin, Beclomethasone, Buprenorphine, Methadone, Atovaquone, Ranolazine or combinations thereof. In some embodiments, the active agents are coated with at least two layers of the high surface coverage chemical vapor deposition polymer coat that can include two or more layers of different coatings and/or different thicknesses. Some of the monomers include ethylene, vinyl alcohol, acrylic acid, carbophil, ethylene glycol, glycolic acid, saccharide, lactic acid, esters, ortho esters, phosphazenes, anhydrides, amides, perfluoroalkenes or a combination thereof. Other monomers include hexamethyldisiloxane, perfluorohexane, methacrylic monomers selected from methacrylic acid (MAA), methyl methacrylate (MMA), poly(methacylic acid)-co-poly(methyl methacrylate) (PMAA-co-PMMA), 2,3,5-trimethyl-3-hexene, 2,3,5-trimethyl-2-hexene, 2,4,5-trimethyl-2-hexene, perfluoroalkane monomers selected from CnF(2n+2) monomers like C2F6, C3F8, C4F10, C5F12, C6F14, C9F18, C6F12, C7F14, and C8F16 or combinations thereof.

Supersaturation in the present invention refers to a solution that contains more of the dissolved materials than could be dissolved by the solvent under normal circumstances.

Surfactant in the present invention refers to a surface acting agent or wetting agent that acts to lower the interfacial tension between two liquids or a liquid and a solid phase.

The gastrointestinal tract is responsible for ingestion, digestion, absorption and waste elimination. The stomach is part of the upper gastrointestinal tract. The intestinal tract, or lower gastrointestinal tract, comprises the small intestine and large intestine. A majority of digestion and absorption of food or drugs, for example, occur in the small intestine. Enteric refers to the small intestine; enteric behavior refers to coatings that promote the release of medication in the small intestine.

Bioavailability is a measurement of the extent of a therapeutically active drug that reaches the systemic circulation and is available at the site of action.

Particles of the present invention are comprised of organic or inorganic molecules. Particles are surrounded or coated by at least one layer of a coating material. Generally, preferred particles are those that remain functional after coating. Functional particles may undergo some structural alterations during coating; however, their general function remains.

Agents of the present invention may be active pharmaceutical ingredients (APIs) or drugs, API/excipient mixtures, nutraceuticals, allergens, botanicals, enzymes, proteins, peptides, carbonaceous compounds, nucleic acids, vitamins, minerals, elemental molecules, fatty acids, lipids, photolabile compounds, food, cosmetics, or dyes, as examples. Agents can be used in various combinations.

Active pharmaceuticals ingredients Active Pharmaceutical Ingredients (APIs) may include analgesic anti-inflammatory agents such as, acetaminophen, aspirin, salicylic acid, methyl salicylate, choline salicylate, glycol salicylate, l-menthol, camphor, mefenamic acid, fluphenamic acid, indomethacin, diclofenac, alclofenac, ibuprofen, ketoprofen, naproxene, pranoprofen, fenoprofen, sulindac, fenbufen, clidanac, flurbiprofen, indoprofen, protizidic acid, fentiazac, tolmetin, tiaprofenic acid, bendazac, bufexamac, piroxicam, phenylbutazone, oxyphenbutazone, clofezone, pentazocine, mepirizole, and the like.

In addition to the Biopharmaceutics Classification System (BCS) Class II compositions and moisture sensitive agents, other active agents may be used including antiinfectives, analgesic and anti-inflammatory agents e.g., NSAIDs, aspirin and other salicylates.

The compounds of this invention may also be administered in the methods of this invention with analgesic and anti-inflammatory agents e.g., NSAIDs and aspirin and other salicylates. Examples of useful agents include ibuprofen, naproxen, sulindac, diclofenac, piroxicam, ketoprofen, diflunisal, nabumetone, etodolac, oxaprozin, indomethacin, melicoxam, valdecoxib, acetaminophen and eterocoxib.

Other compounds having analgesic effects that may be utilized in the method of the present invention include aspirin and other salicylates, acetaminophen, paracetamol, indomethacin, cholinergic analgesics, adrenergic agents, nonsteroidal anti-inflammatory drugs, and other like compounds known in the art.

NSAIDs are well known to those skilled in the art and can be used in their known dosages and dosage regimens. Examples of NSAIDs include but are not limited to: piroxicam, ketoprofen, naproxen, indomethacin, and ibuprofen.

Antiinfectives are agents that act against infections, e.g., bacterial, mycobacterial, fungal, viral or protozoal infections. Antiinfectives covered by the invention include but are not limited to aminoglycosides (e.g., streptomycin, gentamicin, tobramycin, amikacin, netilmicin, kanamycin, and the like), tetracyclines (e.g., chlortetracycline, oxytetracycline, methacycline, doxycycline, minocycline and the like), sulfonamides (e.g., sulfanilamide, sulfadiazine, sulfamethoxazole, sulfisoxazole, sulfacetamide, and the like), paraminobenzoic acid, diaminopyrimidines (e.g., trimethoprim, often used in conjunction with sulfamethoxazole, pyrazinamide, and the like), quinolones (e.g., nalidixic acid, cinoxacin, ciprofloxacin and norfloxacin and the like), penicillins (e.g., penicillin G, penicillin V, ampicillin, amoxicillin, bacampicillin, carbenicillin, carbenicillin indanyl, ticarcillin, azlocillin, mezlocillin, piperacillin, and the like), penicillinase resistant penicillin (e.g., methicillin, oxacillin, cloxacillin, dicloxacillin, nafcillin and the like), first generation cephalosporins (e.g., cefadroxil, cephalexin, cephradine, cephalothin, cephapirin, cefazolin, and the like), second generation cephalosporins (e.g., cefaclor, cefamandole, cefonicid, cefoxitin, cefotetan, cefuroxime, cefuroxime axetil; cefmetazole, cefprozil, loracarbef, ceforanide, and the like), third generation cephalosporins (e.g., cefepime, cefoperazone, cefotaxime, ceftizoxime, ceftriaxone, ceftazidime, cefixime, cefpodoxime, ceftibuten, and the like), other beta-lactams (e.g., imipenem, meropenem, aztreonam, clavulanic acid, sulbactam, tazobactam; and the like), betalactamase inhibitors (e.g., clavulanic acid), chlorampheriicol, macrolides (e.g., erythromycin, azithromycin, clarithromycin, and the like), lincomycin, clindamycin, spectinomycin, polymyxin B, polymixins (e.g., polymyxin A, B, C, D, E1 (colistin A), or E2, colistin B or C, and the like) colistin, vancomycin, bacitracin, isoniazid, rifampin, ethambutol, ethionamide, aminosalicylic acid, cycloserine, capreomycin, sulfones (e.g., dapsone, sulfoxone sodium, and the like), clofazimine, thalidomide, or any other antibacterial agent that can be lipid encapsulated. Antiinfectives can include antifungal agents, including polyene antifungals (e.g., amphotericin B, nystatin, natamycin, and the like), flucytosine, imidazoles (e.g., n-ticonazole, clotrimazole, econazole, ketoconazole, and the like), triazoles (e.g., itraconazole, fluconazole, and the like), griseofulvin, terconazole, butoconazole ciclopirax, ciclopirox olamine, haloprogin, tolnaftate, naftifine, terbinafine, or any other antifungal that can be lipid encapsulated or complexed.

In addition, the present invention provides the ability to provide multiple layers to a substrate with the layer's having a different thickness and/or a different composition. In some instances the coating may be alternating in composition.

Drugs having an action on the central nervous system, for example sedatives, hypnotics, antianxiety agents, analgesics and anesthetics, such as, chloral, buprenorphine, naloxone, haloperidol, fluphenazine, pentobarbital, phenobarbital, secobarbital, amobarbital, cyclobarbital, codeine, lidocaine, tetracaine, dyclonine, dibucaine, cocaine, procaine, mepivacaine, bupivacaine, etidocaine, prilocalne, benzocaine, fentanyl, nicotine, and the like. Local anesthetics such as, benzocaine, procaine, dibucaine, lidocaine, and the like.

Antihistaminics or antiallergic agents such as, diphenhydramine, dimenhydrinate, perphenazine, triprolidine, pyrilamine, chlorcyclizine, promethazine, carbinoxamine, tripelennamine, brompheniramine, hydroxyzine, cyclizine, meclizine, clorprenaline, terfenadine, chlorpheniramine, and the like. Anti-allergenics such as, antazoline, methapyrilene, chlorpheniramine, pyrilamine, pheniramine, and the like. Decongestants such as, phenylephrine, ephedrine, naphazoline, tetrahydrozoline, and the like.

Antipyretics such as, aspirin, salicylamide, non-steroidal anti-inflammatory agents, and the like. Antimigrane agents such as, dihydroergotamine, pizotyline, and the like. Acetonide anti-inflammatory agents, such as hydrocortisone, cortisone, dexamethasone, fluocinolone, triamcinolone, medrysone, prednisolone, flurandrenolide, prednisone, halcinonide, methylprednisolone, fludrocortisone, corticosterone, paramethasone, betamethasone, ibuprofen, naproxen, fenoprofen, fenbufen, flurbiprofen, indoprofen, ketoprofen, suprofen, indomethacin, piroxicam, aspirin, salicylic acid, diflunisal, methyl salicylate, phenylbutazone, sulindac, mefenamic acid, meclofenamate sodium, tolmetin, and the like. Muscle relaxants such as, tolperisone, baclofen, dantrolene sodium, cyclobenzaprine.

Steroids such as, androgenic steroids, such as, testosterone, methyltestosterone, fluoxymesterone, estrogens such as, conjugated estrogens, esterified estrogens, estropipate, 17-β estradiol, 17-β estradiol valerate, equilin, mestranol, estrone, estriol, 17β ethinyl estradiol, diethylstilbestrol, progestational agents, such as, progesterone, 19-norprogesterone, norethindrone, norethindrone acetate, melengestrol, chlormadinone, ethisterone, medroxyprogesterone acetate, hydroxyprogesterone caproate, ethynodiol diacetate, norethynodrel, 17-α hydroxyprogesterone, dydrogesterone, dimethisterone, ethinylestrenol, norgestrel, demegestone, promegestone, megestrol acetate, and the like.

Respiratory agents such as, theophilline and β2-adrenergic agonists, such as, albuterol, terbutaline, metaproterenol, ritodrine, carbuterol, fenoterol, quinterenol, rimiterol, solmefamol, soterenol, tetroquinol, and the like. Sympathomimetics such as, dopamine, norepinephrine, phenylpropanolamine, phenylephrine, pseudoephedrine, amphetamine, propylhexedrine, arecoline, and the like.

Antimicrobial agents including antibacterial agents, antifungal agents, antimycotic agents and antiviral agents; tetracyclines such as, oxytetracycline, penicillins, such as, ampicillin, cephalosporins such as, cefalotin, aminoglycosides, such as, kanamycin, macrolides such as, erythromycin, chloramphenicol, iodides, nitrofrantoin, nystatin, amphotericin, fradiomycin, sulfonamides, purrolnitrin, clotrimazole, itraconazole, miconazole chloramphenicol, sulfacetamide, sulfamethazine, sulfadiazine, sulfamerazine, sulfamethizole and sulfisoxazole; antivirals, including idoxuridine; clarithromycin; and other anti-infectives including nitrofurazone, and the like.

Antihypertensive agents such as, clonidine, α-methyldopa, reserpine, syrosingopine, rescinnamine, cinnarizine, hydrazine, prazosin, and the like. Antihypertensive diuretics such as, chlorothiazide, hydrochlorothrazide, bendoflumethazide, trichlormethiazide, furosemide, tripamide, methylclothiazide, penfluzide, hydrothiazide, spironolactone, metolazone, and the like. Cardiotonics such as, digitalis, ubidecarenone, dopamine, and the like. Coronary vasodilators such as, organic nitrates such as, nitroglycerine, isosorbitol dinitrate, erythritol tetranitrate, and pentaerythritol tetranitrate, dipyridamole, dilazep, trapidil, trimetazidine, and the like. Vasoconstrictors such as, dihydroergotamine, dihydroergotoxine, and the like. β-blockers or antiarrhythmic agents such as, timolol pindolol, propranolol, and the like. Humoral agents such as, the prostaglandins, natural and synthetic, for example PGE1, PGE2α, and PGF2α, and the PGE1 analog misoprostol. Antispasmodics such as, atropine, methantheline, papaverine, cinnamedrine, methscopolamine, and the like.

Calcium antagonists and other circulatory organ agents, such as, aptopril, diltiazem, nifedipine, nicardipine, verapamil, bencyclane, ifenprodil tartarate, molsidomine, clonidine, prazosin, and the like. Anti-convulsants such as, nitrazepam, meprobamate, phenyloin, and the like. Agents for dizziness such as, isoprenaline, betahistine, scopolamine, and the like. Tranquilizers such as, reserpine, chlorpromazine, and antianxiety benzodiazepines such as, alprazolam, chlordiazepoxide, clorazeptate, halazepam, oxazepam, prazepam, clonazepam, flurazepam, triazolam, lorazepam, diazepam, and the like.

Antipsychotics such as, phenothiazines including thiopropazate, chlorpromazine, triflupromazine, mesoridazine, piperracetazine, thioridazine, acetophenazine, fluphenazine, perphenazine, trifluoperazine, and other major tranquilizers such as, chlorprathixene, thiothixene, haloperidol, bromperidol, loxapine, and molindone, as well as, those agents used at lower doses in the treatment of nausea, vomiting, and the like.

Drugs for Parkinson's disease, spasticity, and acute muscle spasms such as levodopa, carbidopa, amantadine, apomorphine, bromocriptine, selegiline (deprenyl), trihexyphenidyl hydrochloride, benztropine mesylate, procyclidine hydrochloride, baclofen, diazepam, dantrolene, and the like. Respiratory agents such as, codeine, ephedrine, isoproterenol, dextromethorphan, orciprenaline, ipratropium bromide, cromglycic acid, and the like. Non-steroidal hormones or antihormones such as, corticotropin, oxytocin, vasopressin, salivary hormone, thyroid hormone, adrenal hormone, kallikrein, insulin, oxendolone, and the like.

Vitamins such as, vitamins A, B, C, D, E and K and derivatives thereof, calciferols, mecobalamin, and the like for dermatologically use. Enzymes such as amylase, trypsin, lipase, and combinations thereof, as well as lysozyme, urokinaze, and the like. Herb medicines or crude extracts such as, Aloe vera, and the like.

Antitumor agents such as, 5-fluorouracil and derivatives thereof, krestin, picibanil, ancitabine, cytarabine, and the like. Anti-estrogen or anti-hormone agents such as, tamoxifen or human chorionic gonadotropin, and the like. Miotics such as pilocarpine, and the like.

Cholinergic agonists such as, choline, acetylcholine, methacholine, carbachol, bethanechol, pilocarpine, muscarine, arecoline, and the like. Antimuscarinic or muscarinic cholinergic blocking agents such as, atropine, scopolamine, homatropine, methscopolamine, homatropine methylbromide, methantheline, cyclopentolate, tropicamide, propantheline, anisotropine, dicyclomine, eucatropine, and the like.

Mydriatics such as, atropine, cyclopentolate, homatropine, scopolamine, tropicamide, eucatropine, hydroxyamphetamine, and the like. Psychic energizers such as 3-(2-aminopropy)indole, 3-(2-aminobutyl)indole, and the like.

Antidepressant drugs such as, isocarboxazid, phenelzine, tranylcypromine, imipramine, amitriptyline, trimipramine, doxepin, desipramine, nortriptyline, protriptyline, amoxapine, maprotiline, trazodone, and the like.

Anti-diabetics such as, insulin, and anticancer drugs such as, tamoxifen, methotrexate, and the like.

Anorectic drugs such as, dextroamphetamine, methamphetamine, phenylpropanolamine, fenfluramine, diethylpropion, mazindol, phentermine, and the like.

Anti-malarials such as, the 4-aminoquinolines, alphaminoquinolines, chloroquine, pyrimethamine, and the like.

Anti-ulcerative agents such as, misoprostol, omeprazole, enprostil, and the like. Antiulcer agents such as, allantoin, aldioxa, alcloxa, N-methylscopolamine methylsulfate, and the like. Antidiabetics such as insulin, and the like.

Anti-cancer agent such as, cis-platin, actinomycin D, doxorubicin, vincristine, vinblastine, etoposide, amsacrine, mitoxantrone, tenipaside, taxol, colchicine, cyclosporin A, phenothiazines or thioxantheres, and the like.

For use with vaccines, one or more antigens, such as, natural, heat-killer, inactivated, synthetic, peptides and even T cell epitopes (e.g., GADE, DAGE, MAGE, etc.) and the like.

Example therapeutic or active agents also include water soluble or poorly soluble drug of molecular weigh from 40 to 1,100 including the following: Hydrocodone, Lexapro, Vicodin, Effexor, Paxil, Wellbutrin, Bextra, Neurontin, Lipitor, Percocet, Oxycodone, Valium, Naproxen, Tramadol, Ambien, Oxycontin, Celebrex, Prednisone, Celexa, Ultracet, Protonix, Soma, Atenolol, Lisinopril, Lortab, Darvocet, Cipro, Levaquin, Ativan, Nexium, Cyclobenzaprine, Ultram, Alprazolam, Trazodone, Norvasc, Biaxin, Codeine, Clonazepam, Toprol, Zithromax, Diovan, Skelaxin, Klonopin, Lorazepam, Depakote, Diazepam, Albuterol, Topamax, Seroquel, Amoxicillin, Ritalin, Methadone, Augmentin, Zetia, Cephalexin, Prevacid, Flexeril, Synthroid, Promethazine, Phentermine, Metformin, Doxycycline, Aspirin, Remeron, Metoprolol, Amitriptyline, Advair, Ibuprofen, Hydrochlorothiazide, Crestor, Acetaminophen, Concerta, Clonidine, Norco, Elavil, Abilify, Risperdal, Mobic, Ranitidine, Lasix, Fluoxetine, Coumadin, Diclofenac, Hydroxyzine, Phenergan, Lamictal, Verapamil, Guaifenesin, Aciphex, Furosemide, Entex, Metronidazole, Carisoprodol, Propoxyphene, Digoxin, Zanaflex, Clindamycin, Trileptal, Buspar, Keflex, Bactrim, Dilantin, Flomax, Benicar, Baclofen, Endocet, Avelox, Lotrel, Inderal, Provigil, Zantac, Fentanyl, Premarin, Penicillin, Claritin, Reglan, Enalapril, Tricor, Methotrexate, Pravachol, Amiodarone, Zelnorm, Erythromycin, Tegretol, Omeprazole, and Meclizine.

The drugs mentioned above may be used in combination as required. Moreover, the above drugs may be used either in the free form or, if capable of forming salts, in the form of a salt with a suitable acid or base. If the drugs have a carboxyl group, their esters may be employed.

An excipient is an inactive substance used as a carrier for the active ingredients of a medication. Excipients can be used as fillers, inert diluents, lubricants, dispersing agents, binders, moisture absorbents, binder-disintegrants, glidants, flow agents, hardness agents, colorants, flavors, anti-adhesives, and various combinations there of. In many cases, an “active” substance (such as aspirin) may not be easily administered and absorbed by the human body; in such cases the substance in question may be dissolved into or mixed with an excipient. Excipients are also sometimes used to bulk up formulations with very potent active ingredients, to allow for convenient and accurate dosage. In addition to their use in the single-dosage quantity, excipients can be used in the manufacturing process to aid in the handling of the active substance concerned. Depending on the route of administration, and form of medication, different excipients may be used. For oral administration tablets and capsules are used. Suppositories are used for rectal administration.

Often, once an active ingredient has been purified, it cannot stay in purified form for long. In many cases it will denature, fall out of solution, or stick to the sides of the container. To stabilize the active ingredient, excipients are added, ensuring that the active ingredient stays “active”, and, just as importantly, stable for a sufficiently long period of time that the shelf-life of the product makes it competitive with other products. Thus, the formulation of excipients in many cases is considered a trade secret.

Pharmaceutical codes require that all ingredients in drugs, as well as their chemical decomposition products are identified and guaranteed to be safe. For this reason, excipients are only used when absolutely necessary and in the smallest amounts possible.

In various embodiments of the present invention, an API/excipient mixture, such as Itraconazole (ITZ) and Eudragit L100-55 excipient, may be used as the agent. The API/excipient mixture may be amorphous or in crystal form. Itraconazole, shown below as structure (d) may be detected by UV-visible spectroscopy and is available in amorphous or crystal form. While uniformly sized particles may be used with the present invention, it is not necessary. In some instances, particles of different sizes may be preferred. In one aspect of the present invention, the agent or core may have a D50 in the range of less than 100 nanometers up to several 100 microns in diameter. D50 is a means of denoting average particle size, and is defined as the diameter where 50 wt % of the particles of a given sample have a larger equivalent diameter, and the remaining 50 wt % of the same given sample of particles have a smaller equivalent diameter.

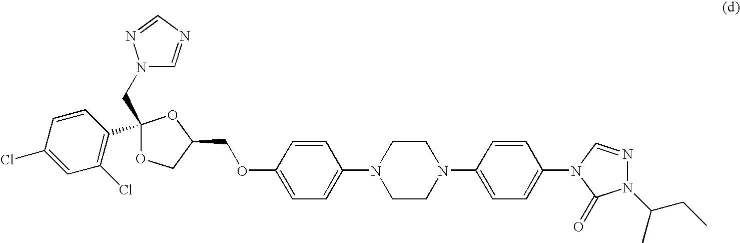

In other embodiments, a pharmaceutical composition, for example aspirin and/or ibuprofen, may be used as the particle. Aspirin, chemically referred to as acetyl salicylic acid, is an antipyretic, anti-inflammatory analgesic with a carboxylic acid backbone group rendering the molecule soluble in various solvents. Acetyl salicylic acid, shown below as structure (e), may be detected by UV-visible spectroscopy and is available in crystal form. While uniformly sized particles may be used with the present invention, it is not necessary. In some instances, particles of different sizes may be preferred. For crystals such as aspirin, uniformity may be obtained by grinding and sieving the crystals followed by drying under vacuum (e.g., 100 degrees Centigrade overnight).

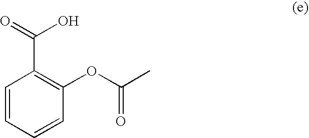

Ibuprofen, chemically referred to as 4-isobutyl-a-methylphenylacetic acid, is an acidic, non-steroidal, anti-inflammatory composition with limited solubility in low pH (<7) solutions and high solubility at higher pH (>7) solutions. Ibuprofen has a carboxylic acid backbone group as shown in structure (f) and may be detected by UV-visible spectroscopy. For ibuprofen, crystals may be sieved and used without drying.

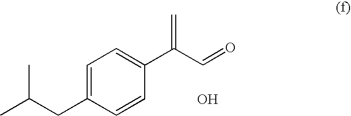

Coating materials of the present invention are used to prepare coatings that encapsulate agents. Coating materials are monomers or carbonaceous compounds (molecules containing at least one carbon) that, upon polymerization (e.g., by deposition), yield polymers or polymer films that are degradable or nondegradable. In many instances, monomers are carbonaceous compounds capable of forming at least one polymer or polymer film degradable by chemical and/or physical processes. Degradation of the polymer or polymer film is then dependant, in part, on the encapsulation process, as described herein. Monomers are also carbonaceous compounds capable of forming at least one polymer or polymer film that is not degradable. As such, the encapsulating polymer or polymer film is capable of releasing the particle via one or more processes, such as dissolution of all or a portion of the particle, chemical degradation of the encapsulating polymer, physical degradation of the encapsulating polymer, and/or passage of the all or a portion of the particle through the polymer (e.g., through pores, spaces, or openings in the polymer or polymer film). Release of a particle encapsulated by such a degradable or nondegradable polymer is also dependent, in part, on the encapsulation process, as described herein.

Degradable polymers include natural polymers (e.g., polysaccharides) as well as synthetic polymers, which are easy to manipulate (e.g., polyesters, polyanhydrides, polyamides, phosphorous-containing polymers). Examples of degradable coatings or polymer films prepared by the present invention are listed in TABLE 1. The coating materials that form such coatings or polymers are the monomeric subunits. Examples of these monomeric subunits include ethylene, vinyl alcohol, acrylic acid, carbophil, ethylene glycol, glycolic acid, saccharide, lactic acid, esters, ortho esters, phosphazenes, anhydrides, amides, as examples.

TABLE 1
Coating Materials of the Present Invention

| Backbone Structure | Coating Material | Comments |
|---|---|---|
| C—C | Polyethylene (PE) | Zero-order temporal control achieved by diffusion |
| | | from matrices. |
| Vinyl-based C—C | Poly(vinyl alcohol) (PVA) | Bioadhesive hydrogels. Surface stabilizer in |
| | | microsphere formulation. |
| | Poly(acrylic acid) (PAA) | Bioadhesive polymer. Hydrogels of PAA reversibly |
| | | swell as a function of pH. |
| | Polycarbophil | Hydrogels. Mucoadhesive properties allow |
| | | temporal and distribution control. |
| C—O | Polyethylene glycol (PEG) | Used as diffusion-limited tablet formulation, |
| | | cross-linked hydrogels and polymer conjugates. |
| C—O, C═O | Poly(glycolic acid) (PGA) | Biosynthetic poly(esters) used in the formulation |
| | | of matrices containing human growth hormone. |
| | Poly(lactic acid) (PLA) | |
| | Poly(ortho esters) | Degradable polymers. Number of applications of |
| | | 3,9-diethylidene-2,4,8,10-tetraoxaspiro[5.5]undecane |
| | | (DETOSU)-based poly(ortho esters). |
| | Poly(anhydrides) | Heterogeneous surface erosion. Polyanhydride |
| | | matrices used in microencapsulation of insulin, |
| | | enzymes and growth factors. |
| Phosphorous-based | Poly(phosphazenes) | Amino acid side chains generate flexible materials |
| P═N, P—O | | that degrade to amino acid, phosphate, and |
| | | ammonia poly[bis(glycine ethyl ester)phosphazene]. |

A degradable polymer generally releases its encapsulated particle into an environment through a process that includes degradation of the encapsulating polymer. A degradable polymer, as with a nondegradable polymer, may also have pores, spaces, or openings through which all or portions of the particle may pass.

Degradation of a degradable polymer generally occurs via bond cleavage and/or erosion. For biologic systems, degradation often occurs via enzymatic cleavage or hydrolysis, in which the polymer backbone is cut using a chemical process. With erosion, a physical process occurs, generally involving surface erosion or bulk erosion.

One feature of the present invention is that degradation of a polymer or polymer film may be controlled. Similarly, the present invention is capable of controlling other characteristics of a polymer or polymer film that affect particle release. Hence, the present invention is capable of controlling the release of a particle into an environment. Such control occurs because the present invention is capable of altering one or more conditions of the polymer or polymer film. Coating conditions include altering the surface area of a coating, adjusting the cross-linking of the coating material, altering the wetness, hydrophilicity or hydrophobicity of the coating, changing the density of side groups or functional groups in the coating or coating material, and/or altering the overall thickness of the coating. These coating conditions may be altered for an encapsulation process involving degradable and/or nondegradable polymers. In some instances, encapsulation may include more than one polymer.

In one aspect of the present invention, coating materials such as perfluorohexane (C6F14), methyl methacrylate (MMA), and methacrylic acid (MAA) are provided. Coatings or polymer films obtained by plasma polymerization of methacrylic acid and methyl methacrylate are hydrophilic. Coatings or polymer films obtained by plasma polymerization of perfluorohexane are hydrophobic. Chemical structures of (a) perfluorohexane, (b) methyl methacrylate, and (c) methacrylic acid are shown below.

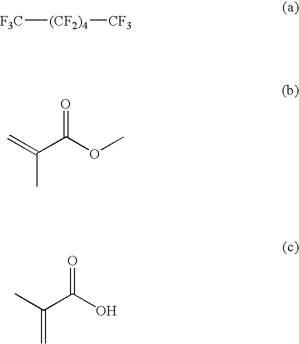

In another aspect of the present invention, the coating layer of the particle was designed for release of the API/excipient in an environment of pH 6.8 (to simulate intestinal fluid). Testing was conducted for two hours in 0.1 N HCl solution (to simulate gastric fluid) wherein the coating layer, PMAA-co-PMMA, did not dissolve or degrade. When the pH was then adjusted to 6.8, the coating layer degraded and Itraconazole was released into the solution.

In another aspect of the present invention, the coating layer of the particle was designed to allow supersaturation of the API/excipient in an environment of pH 6.8 (to simulate intestinal fluid). Testing was conducted for two hours in 0.1 N HCl solution (to simulate gastric fluid) followed by a switch to pH 6.8 which caused the coating layer, PMAA-co-PMMA, to rapidly degrade. When the pH was then adjusted to 6.8, the coating layer degraded and Itraconazole was released into the solution at a supersaturated level.

In yet another aspect of the present invention, the coating layer of the particle was designed to allow release and supersaturation of the API/excipient in an acidic environment similar to that of the stomach (0.1 N HCl). Testing was conducted for two hours in 0.1 N HCl solution which caused the coating layer to rapidly degrade and release Itraconazole at a supersaturated level.

In yet another aspect of the present invention is acid labile so that the coating layer of the particle is designed to protect the particle and in an acidic environment similar to that of the stomach allow for enteric compounds that are generally resistant to disintegration in acidic media but will begin to disintegrate in solutions with pH greater than 4.5. Similarly, the coating of the instant invention can be used as a moisture barrier to protect the compositions from decomposition or reaction with moisture or other agents. As a result, a variety of labile compositions may be used in conjunction with the instant invention.

Many pharmaceutical compounds are susceptible to degradation in acidic environments. For example, antibiotics such as erythromycin; proton pump inhibitors (or “PPIs”) such as lansoprazole, or omeprazole; and pencreatin; are compounds that degrade in acidic environments and are therefore referred to as “acid labile”. Oral delivery of acid labile pharmaceutical compounds is challenging because the gastric pH is very acidic (typically between about pH 1.5 and 1.9). Under gastric conditions, acid-labile drugs typically degrade and are not readily available for uptake without being protected. Due to the pH sensitivity of acid labile drugs, they typically are administered in a form that protects the drug from the acidic gastric environment. Enteric coatings are probably the most widely used method of protecting acid-labile drugs from gastric degradation. Enteric coating methods typically form a barrier around drug particles, or an entire dosage form containing an acid-labile drug, with a coating that does not dissolve upon introduction to the low pH of the gastric environment. Such enteric coatings typically dissolve at a pH greater than 6, such as that found in the upper small intestine where the acid labile drugs are released in an environment where they will not significantly degrade, and therefore can be absorbed. Drugs requiring enteric coatings are often times formulated as capsules or tablets that are difficult to administer to patients who have difficulty swallowing such as pediatric patients, or patients who cannot swallow at all such as critically ill patients. The present invention provides for preferentially absorbed in upper and/or lower intestine.

Another embodiment of the invention is to provide a coating layer for enteric compounds that are generally resistant to disintegration in an acidic environment such as the stomach, but predisposed to dissolve at pH 5.5 or higher as in the intestinal tract. Targeted delivery of the encapsulated particle and release of the API or API/excipient mixture therefrom into the intestinal tract is thus controlled by the coating layer.

With the present invention, the carbonaceous compound may be pretreated before use. For allyl alcohol—an oxygen containing organic monomer that is very soluble at pHs ranging from 1 to 10—as well as perfluorohexane (C6F14)—a perfluorocarbon compound that is sparingly soluble at pHs ranging from 1 to 10—the compounds were degassed by freeze-thaw cycles before use. A similar procedure was also performed for methyl methacrylate and methacrylic acid. Each carbonaceous compound is also handled in the proper manner based on its chemical composition, as is well known in the art. For examples, allyl alcohol and perfluorohexane were protected from light and stored at room temperature, while methyl methacrylate was protected from light and stored in the refrigerator at 4 degrees Centigrade.

Perfluorocarbon compounds, such as perfluorohexane, yield plasma polymerized fluorinated films that exhibit good adhesion to many organic and inorganic substrates, have low intermolecular forces, low friction coefficient, and are biocompatible. Perfluorocarbon compounds of any composition may be used in the present invention. For example the individual monomers may be combined and reacted to form the fluoropolymer. For example, two specific fluorinated monomers are C6F14 and C9F18; however, others linear perfluoroalkane monomers with the formula CnF(2n+2) like C2F6, C3F8, C4F10, C5F12, C6F14, etc. In addition, cyclic perfluoroalkanes like C6F12, C7F14, and C8F16 may also be used. Finally perfluoroalkenes or those molecules containing carbon double bonds like C9F18 which is a mixture of three perfluoro compounds (2,3,5-trimethyl-3-hexene, 2,3,5-trimethyl-2-hexene, 2,4,5-trimethyl-2-hexene) may also be used.

The present inventors have previously shown that a pulsed plasma polymerization process may be used with perfluorocarbon compounds to create polymers and polymers films. (See U.S. Pat. No. 5,876,753; U.S. Pat. No. 6,306,506; U.S. Pat. No. 6,214,423; all of which are herein incorporated by reference). Polymers of hexafluoro-propylene oxide (C3F6O), perfluoro-2-butyltetrahydrofuran (PF2BTHF, C8F16O) and perfluoropropylene (C3F6) create excellent coatings or films that are capable of attaching to substrate surfaces. The preferred perfluoro compound is a perfluorocarbon e.g., the most preferred perfluorinated trifluoromethyl substituted perfluorohexene. To form a perfluorinated surface also having a reactive surface, a perfluorinated compound is mixed with a carbonaceous compound having a reactive functional group e.g., an alkenyl or alkyl halide, isothiocyanate, cyanide, benzene, acetate, mercaptan, glycidyl ether, ether, chloroformate, methyl sulfide, phenyl sulfone, phosphonic dichloride, trimethylsilane, triethoxysilane, acid, acid halide, amine, alcohol, or phosphide. The target materials may include any substance capable of reacting with the reactive functional groups. Preferred target materials include amino acids, enzymes, fluorinated amino acids, proteins, peptides, saccharides, hormones, hormone receptors, polynucleotides, oligonucleotides, carbohydrates, glycosaminoglycans (e.g., heparin, for example) polyethylene glycol and polyethylene oxide. Derivatives of all these various target materials may be prepared and still retain reactivity with one or more of the active functional groups such that they may be attached to an activated surface. In one aspect the present invention involves producing a surface with reduced adherence for biological materials. Surfaces with coupled polyethylene glycol, polyethylene oxide or abundant —CF3 groups are among the most preferred substituents for producing a surface with increased moisture protection, hydrophobicity and general stability. The use of a highly —CF3 substituted fluorocarbon monomer can yield exceptionally hydrophobic (or stable) surfaces via plasma deposition. For example, utilizing low duty cycle RF plasma deposition it is possible to retain, to a very high degree, the —CF3 content of the starting monomer.

Siloxane compounds, such as Hexamethyldisiloxane (HMDSO), also yield plasma polymerized films that exhibit good adhesion to many organic and inorganic substrates, have low intermolecular forces, low friction coefficient, hydrophobic behavior, and are biocompatible.

Plasma Enhanced Chemical Vapor Depositions PECVD provides for a solventless, pin-hole free, single-step encapsulation process in which the encapsulating or coating material may be modified depending on the process, itself. For example, the process is able to control encapsulation, and hence, particle introduction into an environment, by adjusting the side groups, thickness, wettability, molecular weight, cross-linking density, surface area and/or composition of the coating material.

With the present invention, both pulsed and the more conventional continuous-wave (CW) plasma approaches may be used. For example, the present inventors have shown that using a pulsed plasma approach provides excellent film chemistry control during polymer formation and control of film thickness (Susut C and Timmons R B, Plasma enhanced chemical vapor depositions to encapsulate crystals in thin polymericfilms: a new approach to controlling drug release rates, International Journal of Pharmaceutics, 2004, in press; herein incorporated by reference). Pulsed applications may reduce or eliminate undesirable plasma-induced chemical changes to particles. In addition, under pulsed reaction conditions, significant film formation occurs during plasma off periods (and undesirable high energy reactions between ion-radical and particle are minimized).

Sample Reaction Conditions Using a Pulsed Radio Frequency Plasma Reactor. A 360° rotatable plasma reactor was employed to help achieve uniform and complete coating of particles. However, different reactor types may be used to achieve uniform and complete coating of particles. For example, general agitation methods may be used to achieve uniform and complete coating of particles, e.g., a mill, fixed bed, flat hearth, fluidized bed, vibration bed, spouted bed, circulating fluidized bed, or a drop tower reactor configuration can also be used to help achieve uniform and complete coating of the particles. In one form of the present invention, a cylindrical Pyrex glass reactor of 2.5-60 centimeters internal diameter and 10-250 centimeters in length was used as the plasma chamber. Radio frequency (RF) power to the reactor was provided through two concentric metal rings separated by a distance of approximately 5-240 centimeters. The volatile reaction products and unreacted monomer were collected in a liquid nitrogen cold trap located downstream of the reactor. A butterfly valve controller with pressure transducer (MKS Baratron Model 252A) was used both to monitor and control pressure in the reactor. The flow rate of the monomer was controlled and monitored by a flowmeter placed upstream of the reactor. Ferrofluidic valves, inserted at both ends of the reactor tube, permitted complete rotation of the reactor chamber under vacuum conditions. The rotation rate was controlled with a variable speed motor (Dayton Model 4Z827) connected by pulley to the reactor.

In one embodiment of this invention, the reactor includes a radio frequency amplifier (ENI model A300), a function generator (Tektronix model AFG3102), and a capacitor/inductor matching network used to tune the circuit to minimize reflected power. Applied and reflected power were measured in volts with an oscilloscope (Tektronix model TDS1001B) and in Watts with a wattmeter (Bird model 4421) which were also used to monitor the matching network. The matching network was employed to minimize the reflected energy during the course of each run. The entire reactor was located inside a Faraday cage to prevent radiation of the RF energy to the external environment. While a radio frequency of 13.56 MHz was used, other frequencies may also be used as seen fit or as required.

Coating layers of the present invention were deposited onto agents using a reactor, similar to one described above. Those skilled in the art will appreciate that the features described may also be modified as needed. For most reactions, the rotation rate was kept steady (e.g., 1-120 rev/minute for amorphous Itraconazole). The lower rotation rate for itraconazole minimized the adsorption of the smaller particles on the walls of the reactor chamber by electrostatic forces. The quantity of particles placed in the reaction chamber, in each run, was, in some cases, used as a variable and this effect was evaluated

Self-aggregation and/or electrostatic forces were reduced by several methods, including increasing the monomer flow rate, decreasing the rotation rate of the reactor chamber and/or limiting the peak power to 100 Watts or less. Applying vibration to the reactor walls as well as applying a surface treatment to minimize adhesions may also be employed. In addition, it is also possible to recover coated particles that have adhered to the reactor wall. In general, the percent recovery (ratio of the amount of recovered particles that are coated vs. total amount of particles introduced into the reactor) may typically range from 50% to 99%. One skilled in the art will appreciate that other typical ranges may apply.

Before each coating, the reactor chamber was pre-cleaned (e.g., with soap and water and acetone). It was then vacuumed to a background pressure (e.g., approximately 10 mTorr). Next, the reactor was treated with an oxygen plasma discharge (e.g., 100 Watts at 100 mTorr pressure, operated at a duty cycle of ⅓ ms/ms or ⅕ ms/ms). Pre-cleaning removes polymer residues from the chamber due to previous coatings. After the oxygen plasma discharge, particles would be placed into the reactor. The two ends of the chamber were stoppered (e.g., with glass wool) to keep the particles in the chamber during coating. The reactor chamber was then evacuated to the background pressure.

Plasma Polymerization. In general, and for example coatings provided herein, the reaction chamber was rotated constantly. Using the pulsed plasma approach, significant polymer film formation occurred during plasma off periods, a time when undesirable high energy reactions between ion-radical and particles are minimized. A process of continuous wave plasma polymerization may also be employed to encapsulate particles.

The average power employed under pulsed plasma conditions was calculated according to the formula shown below (1), where τon and τoff are the plasma on and off times and Ppeak is the peak power. By using pulsed plasma polymerization, the average power employed during film formation was often much lower than the power employed under continuous wave reaction conditions, because of the relatively longer plasma off times compared to plasma on times.

Paverage=(τon/(τon+τoff))×Ppeak (1)

Deposition (polymerization) of the coating or polymer film of the present invention was controlled by altering a number of variables associated with the plasma reactor. Variables included duty cycle, power input, peak power, flow rate of the monomer, pressure of the reactor, coating time period and quantity of particles introduced into the reaction chamber at a time.

With the present invention, suitable plasma on/off times (duty cycles) were generally in the millisecond range. As used herein, duty cycles are reported as on/off times per cycle and provided in units of ms/ms. Suitable peak powers ranged from about 5 W to about 300 Watts. Suitable coating periods were typically between about 1 minute and 2 hours. In some cases, self-aggregation of particles may help determine the coating time period. The amount of particles coated at a time typically ranged from about 1 gram to about 100 grams. Flow rates were about 1 sccm to about 100 sccm. The pressure of the reactor typically varied from about 10 mTorr to about 500 mTorr. Those skilled in the art will appreciate that, while typical ranges and values are provided, there is no reason that other values may not be applied, as needed.

To help characterize the coating or polymer film deposited by the present invention, replicate runs of certain carbonaceous compounds were provided in which the carbonaceous compound was deposited on one or more solid substrates, such as silicon wafers and KBr surfaces. The FT-IR spectra were collected with a standard FTIR spectrophotometer operating in both transmission and TR mode typically using 4 cm-1 resolution. Spectra were recorded in absorption mode on polymer films deposited on KBr discs or silicon wafers. The thickness of the films deposited on silicon wafers were measured using a Tencor Alpha Step 200 profilometer. A syringe needle was employed to scribe a scratch on the films. Thickness calculations were based on the difference between the height of the film and original height of the substrate.

Silica gel, polyester-backed Thin-Layer Chromatography TLC plates of thickness 250 fan were used to analyze the separation and/or breakdown of compounds after polymerization and after particle release into an environment. Before use, TLC plates were dried in an oven for about 1 hour at 110 degrees Centigrade to remove adsorbed atmospheric moisture.

For calculations, the distinction between different components in a mixture was determined by a physical constant called retention factor (RF) which is based on the preferential interaction between the compound and the TLC plate. It is known that each compound generally has a different retention factor. If a compound is converted, separated, or structurally altered during plasma polymerization, it will generally have a different RF value. Thus, free particles and encapsulated particles were prepared by dissolving 10 mg of each in 1 ml of dichloromethane.

All TLC solutions were freshly made and aliquots of 5 jA were applied as spots approximately 1 cm apart onto 5×17 cm silica gel TLC plates. A chloroform-acetone (4+1) solvent system was used. Plates were air-dried and analyzed by iodine vapor. Retention factors were calculated for each encapsulated particle and compared to the value obtained for unencapsulated (i.e., free) particle. These values were compared to those known in the literature.

Allyl alcohol was used as a representative carbonaceous compound for coating particles of the present invention. It was determined that as the RF duty cycle was reduced, the retention of the monomer's oxygen content increased, leading to an increase in the hydrophilicity of the coating or polymer film (also referred to herein as film). An increase in the plasma off time also caused an increase in the —OH group incorporation in the coating thus increasing surface density of polar groups. In addition, significant polymer film growth occurred during the plasma off times. Deposition per pulse cycle was shown to increase at constant on time and power, as the off time increased.

### Example 1

### Amorphous Itraconazole Particles with PMAA-co-PMMA Coating

For this study a plasma enhanced chemical vapor deposition process was employed using both methacrylic acid (MAA) and methyl methacrylate (MMA) monomers to deposit a thin coating layer on the surface of an amorphous Itraconazole (ITZ) drug particle. The amorphous ITZ drug particles were prepared using a process known as hot melt extrusion (HME) and were prepared from a pre-extrusion blend of crystalline ITZ and Eudragit L100-55 excipient (1:2). The final potency of the as prepared HME amorphous particles was 28.73% (SD of 0.09%) as measured by high pressure liquid chromatography (HPLC). The coating layer was deposited using a variable duty cycle and variable power 13.56 MHz plasma enhanced chemical vapor deposition coating process. During the first stage of the coating process a peak power of 15 watts and a duty cycle of 5 ms on and 30 ms off was utilized for 60 minutes at a constant reactor pressure of 100 mTorr. After 60 minutes the coating process was switched to a peak power of 22 watts and a duty cycle of 0.5 ms on and 30 ms off for an additional 60 minutes at a constant reactor pressure of 100 mTorr. During both stages of the coating process the monomer flow rates of MAA and MMA were maintained independently at 75 and 25 sccm, respectively. This process yielded a stable and sufficiently adherent gradient layered coating on the HME particles. After coating the amorphous HME particles were tested using differential scanning calorimetry and showed no recrystallization occurred during the coating process. The potency of the particles after coating was also tested and was measured to be 28.62% (SD of 0.17%).

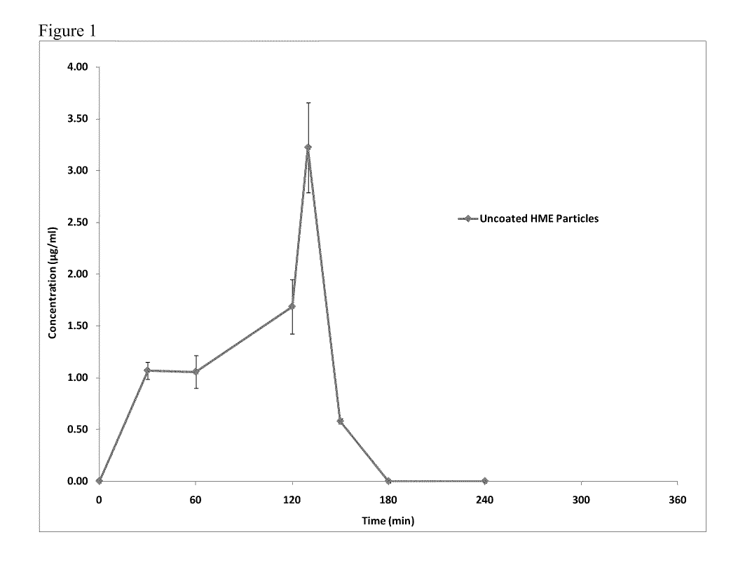

FIG. 1 shows the dissolution testing results for the uncoated amorphous HME control particles. Dissolution testing was performed according to USP 29 Apparatus 2 guidelines (paddle method) at 50 rpm and a constant bath temperature of 37.0+/−0.2° C. In this test 6 mg ITZ equivalent (based on the measured potency), which corresponds to 20 times the saturation solubility of ITZ in acidic media (4 μg/ml)1, was added to each dissolution vessel. Testing was conducted for 2 hrs in 75 ml of 0.1 N HCl followed by a pH-adjustment to 6.8+/−0.5 with the addition of 25 ml of tribasic sodium phosphate solution. The solubility of ITZ in neutral media is considerably lower at (˜1 ng/ml)1. The results shown in FIG. 1 indicate that some dissolution of the uncoated HME particles does occur during the first two hours of testing in the acidic media, which is representative of the conditions within the stomach. After the addition of the tribasic sodium phosphate solution, which is representative of the conditions of the upper small intestine, there is a spike in the measured solubility of the ITZ, at the 130 minute time point, to 3.22 μg/ml which is approximately 3200 times the equilibrium solubility of ITZ in neutral media. After this spike in measured solubility there is a steady decline until the measured level is below the detection limit of the instrument, at the 180 min time point, presumably due to the precipitation of the ITZ out of solution.

Supersaturation in the Intestinal Tract (Coated Particles)

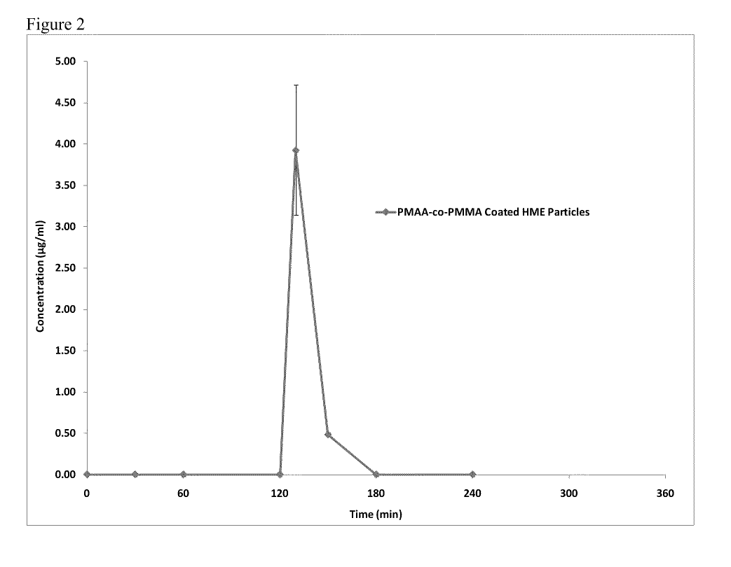

FIG. 2 shows the dissolution testing results for the poly(methacylic acid)-co-poly(methyl methacrylate) (PMAA-co-PMMA) coated amorphous HME particles. The conditions used for the testing of these materials were identical to the ones described above for the testing of the uncoated HME control particles. The results shown in FIG. 2 indicate that no measurable amount of dissolution of the PMAA-co-PMMA coated amorphous HME particles occurs during the first two hours of testing in the acidic media. After the addition of the tribasic sodium phosphate solution there is a spike in the measured solubility of the ITZ, at the 130 minute time point, to 3.93 μg/ml which is approximately 3900 times the equilibrium solubility of ITZ in neutral media. After this spike in measured solubility there is a steady decline until the measured level is below the detection limit of the instrument, at the 180 min time point, presumably due to the precipitation of the ITZ out of solution.

### Example 2

### Amorphous Itraconazole Particles with Perfluorohexane Coating

A plasma enhanced chemical vapor deposition process was employed using perfluorohexane (C6F14) monomer to deposit a thin coating layer on the surface of an amorphous ITZ drug particle. The amorphous ITZ drug particles were prepared using a process known as HME and were prepared from a pre-extrusion blend of crystalline ITZ and Eudragit L100-55 excipient (1:2). The final potency of the as prepared HME amorphous particles was 28.73% (SD of 0.09%) as measured by HPLC. The coating layer was deposited using a 13.56 MHz plasma with a peak power of 150 watts and a duty cycle of 10 ms on and 40 ms off. The reaction chamber was maintained at a pressure of 160 mTorr with a monomer flow rate of 100 sccm for 75 minutes. After coating the amorphous HME particles were tested using differential scanning calorimetry and showed no recrystallization occurred during the coating process. The potency of the particles after coating was also tested and was measured to be 28.67% (SD of 0.09%).

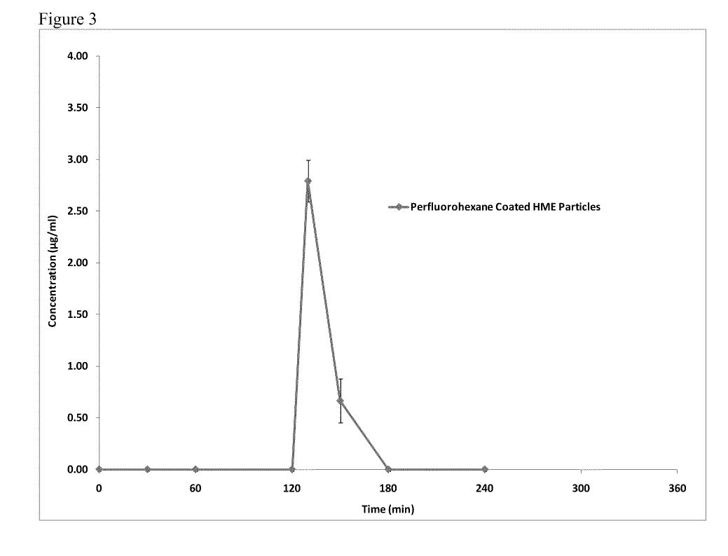

Supersaturation in the Intestinal Tract. FIG. 3 shows the dissolution testing results for the C6F14 coated amorphous HME particles. The conditions used for the testing of these materials were identical to the ones described above for the testing of the uncoated HME control particles. The results shown in FIG. 3 indicate that no measurable amount of dissolution of the C6F14 coated amorphous HME particles occurs during the first two hours of testing in the acidic media. After the addition of the tribasic sodium phosphate solution there is a spike in the measured solubility of the ITZ, at the 130 minute time point, to 2.79 μg/ml which is approximately 2800 times the equilibrium solubility of ITZ in neutral media. After this spike in measured solubility there is a steady decline until the measured level is below the detection limit of the instrument, at the 180 min time point, presumably due to the precipitation of the ITZ out of solution.

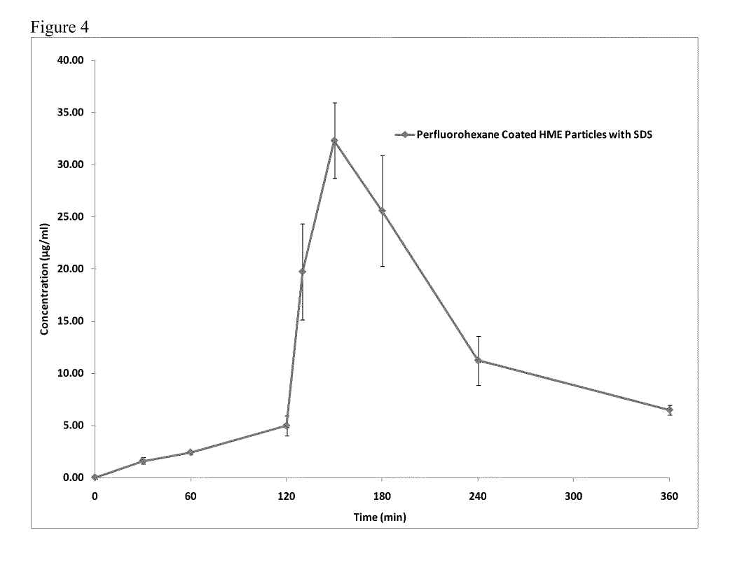

Supersaturation in the Intestinal Tract (Surfactant Added). FIG. 4 shows the dissolution testing results for the C6F14 coated amorphous HME particles with added sodium dodecyl sulfate (SDS) surfactant in the dissolution media. The conditions used for the testing of these materials were identical to the ones described above for the testing of the uncoated HME control particles except for the addition of a small amount of SDS surfactant (conc. of 0.07%) to help ensure wetting of the very hydrophobic perfluorohexane coated amorphous HME particles. The results shown in FIG. 4 indicate that some dissolution of the perfluorohexane coated amorphous HME particles does occur during the first two hours of testing in the acidic media. After the addition of the tribasic sodium phosphate solution there is a spike in the measured solubility of the ITZ, at the 150 minute time point, to 32.28 μg/ml which is approximately 32000 times the equilibrium solubility of ITZ in neutral media. After this spike in measured solubility there is slow decline in the measured solubility level down to 6.47 μg/ml at the 360 minute time point, which is the final time point taken in this study, presumably due to the precipitation of the ITZ out of solution.

### Example 3

### Crystalline Itraconazole Particles with PMAA-co-PMMA Coating

A plasma enhanced chemical vapor deposition process was employed using both MAA and MMA monomers to deposit a thin coating on the surface of a crystalline ITZ drug particle. The crystalline ITZ particles (BP micronized) were purchased from Hawkins, Inc. and used as received. The coating was deposited using a 13.56 MHz plasma with a peak power of 16 watts and a duty cycle of 0.5 ms on and 30 ms off. The reaction chamber was maintained at a pressure of 100 mTorr and the monomer flow rates of MAA and MMA were maintained independently at 75 and 25 sccm, respectively. After coating the ITZ particles were examined using differential scanning calorimetry which confirmed the presence of crystalline drug material. The potency of the ITZ particles after coating was also tested and was measured to be 99.87% (SD of 0.21%).

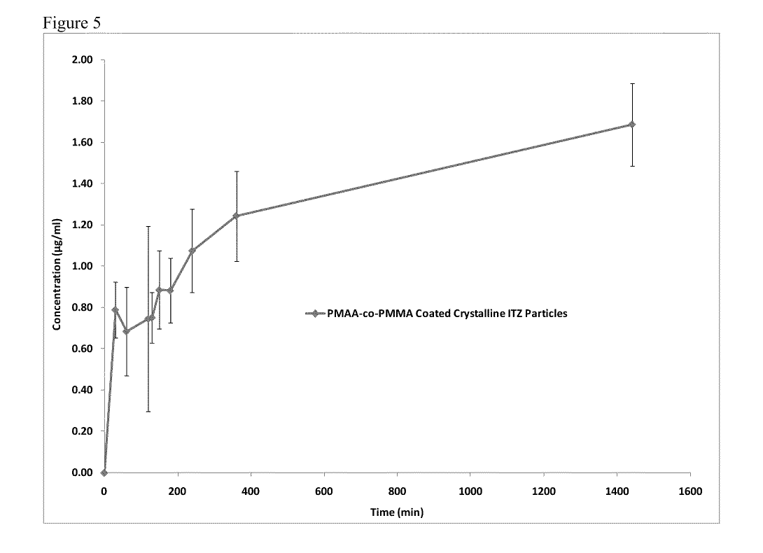

FIG. 5 shows the dissolution testing results for the PMAA-co-PMMA coated crystalline Itraconazole ITZ particles with added SDS surfactant in the dissolution media. Dissolution testing was performed according to USP 29 Apparatus 2 guidelines (paddle method) at 50 rpm in a Vankel 7000 Dissolution Tester (Vankel Technology Group, Cary, N.C.) and a constant bath temperature of 37.0+/−0.2° C. The dissolution method utilized was in accordance with the USP 29 dissolution testing specifications for delayed-release dosage forms Method A. In this test 6 mg ITZ equivalent (based on the measured potency), which corresponds to 20 times the saturation solubility of ITZ in acidic media (4 μg/ml)1, was added to each dissolution vessel. Testing was conducted for 2 hrs in 75 ml of 0.1 N HCl followed by a pH-adjustment to 6.8+/−0.5 with the addition of 25 ml of tribasic sodium phosphate solution. The solubility of ITZ in neutral media is considerably lower at (˜1 ng/ml)1. Additionally, a small amount of SDS surfactant (conc. of 0.07%) was also added to the dissolution media to help ensure wetting of the PMAA-co-PMMA coated particles. The results shown in FIG. 5 indicate that some dissolution of the ITZ particles does occur during the first two hours of testing in the acidic media which is representative of the conditions within the stomach. After the addition of the tribasic sodium phosphate solution, which representative of the conditions of the upper small intestine, there is an increase in the measured solubility of the ITZ, at the 130 minute time point, to 0.75 μg/ml which is approximately 750 times the equilibrium solubility of ITZ in neutral media. After this rise in measured solubility there is a steady increase in the measured solubility level up to 1.69 μg/ml at the 1440 minute time point, which is the final time point taken in this study.

### Example 4

### Crystalline Itraconazole Particles with Perfluorohexane Coating

For this study a plasma enhanced chemical vapor deposition process was employed using C6F14 monomer to deposit a thin coating on the surface of a crystalline ITZ and Eudragit L100 blended drug particle. The blended drug particles were prepared via physical blending in a mortar and pestle with a pre-blend ratio of crystalline ITZ to Eudragit L100 excipient of (1:9). After blending the particles were auto sieved for 60 minutes using a Meinzer II sieve Shaker with seven trays ranging from 250 micron down to 10 micron mesh size cut-offs arranged vertically in series from largest to smallest cut-off size. After sieving the particles were collect in tranches based on particle diameter and then coated. The coating was deposited using a 13.56 MHz plasma with a peak power of 150 watts and a duty cycle of 10 ms on and 40 ms off. The reaction chamber was maintained at a pressure of 160 mTorr with a monomer flow rate of 100 sccm for 75 minutes. After coating the blended particles were examined using differential scanning calorimetry which confirmed the presence of crystalline drug material. The potency of the blended particles after coating was also tested and was measured to be 2.18% (SD of 0.24%).

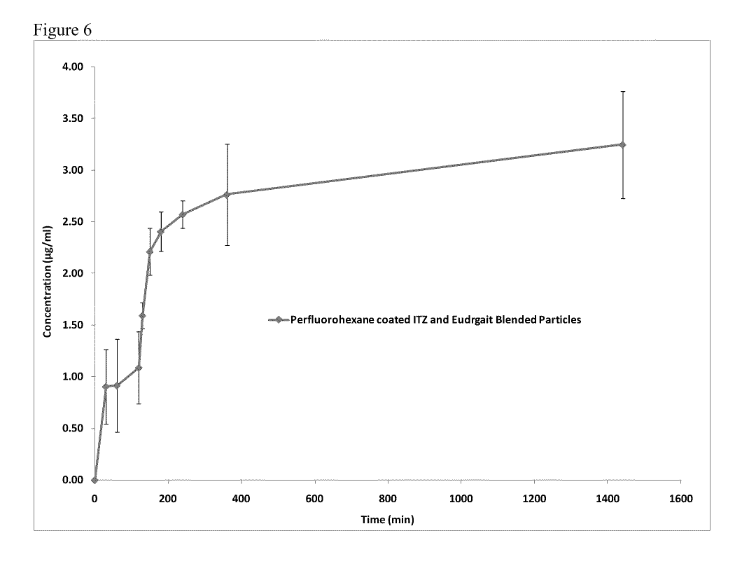

FIG. 6 shows the dissolution testing results for the C6F14 coated crystalline ITZ and Eudragit L100 blended drug particles with added SDS surfactant in the dissolution media. Dissolution testing was performed according to USP 29 Apparatus 2 guidelines (paddle method) at 50 rpm in a Vankel 7000 Dissolution Tester (Vankel Technology Group, Cary, N.C.) and a constant bath temperature of 37.0+/−0.2° C. The dissolution method utilized was in accordance with the USP 29 dissolution testing specifications for delayed-release dosage forms Method A. In this test 6 mg ITZ equivalent (based on the measured potency), which corresponds to 20 times the saturation solubility of ITZ in acidic media (4 μg/ml)1, was added to each dissolution vessel. Testing was conducted for 2 hrs in 75 ml of 0.1 N HCl followed by a pH-adjustment to 6.8+/−0.5 with the addition of 25 ml of tribasic sodium phosphate solution. The solubility of ITZ in neutral media is considerably lower at (˜1 ng/ml)1. Additionally, a small amount of SDS surfactant (conc. of 0.07%) was also added to the dissolution media to help ensure wetting of the very hydrophobic C6F14 coated blended particles. The results shown in FIG. 6 indicate that some dissolution of the blended particles does occur during the first two hours of testing in the acidic media which is representative of the conditions within the stomach. After the addition of the tribasic sodium phosphate solution, which representative of the conditions of the upper small intestine, there is an increase in the measured solubility of the ITZ, at the 130 minute time point, to 1.59 μg/ml which is approximately 1590 times the equilibrium solubility of ITZ in neutral media. After this spike in measured solubility there is a steady increase in the measured solubility level up to 3.25 μg/ml at the 1440 minute time point, which is the final time point taken in this study.

### Example 5

### In-Vivo Animal Models

Institutionally approved in vivo studies were conducted using Sprague-Dawley rats (Charles River Laboratories, Inc., Wilmington, Mass.), which were pre-catheterized with a vascular catheter surgically inserted into the jugular vein. All rats received were between 275 and 325 g of total body weight. After 3 days of acclimatization period, the rats were administered the aqueous dispersion of the formulations by oral gavage at a dose of 30 mg ITZ/kg body weight (n=3). Each formulation was dispersed in a sucrose solution just before dosing such that 400 μL of suspension contained a dose of 9 mg ITZ. Serial blood samples (approximately 0.3 mL each) were withdrawn through the jugular vein catheter at 0, 2, 3, 4, 5, 6, 8, 12, and 24 h after dosing, and placed into a pre-heparinized microcentrifuge tube. Equal volume of saline was replaced after each sampling. Plasma samples were harvested by centrifugation of the blood at 3000×g for 15 min and were kept at −20° C. until drug analysis.

Calibration standards and plasma samples were analyzed according to previously published methods (Gubbins, Gurley, & Bowman; 1998; Vaughn et al., 2006). Briefly, upon thawing, a volume of harvested plasma was transferred to a clean microcentrifuge tube. Barium hydroxide 0.3 N (50 μL) and 0.4 N zinc sulfate heptahydrate solution (50 μL) were then added followed by vortex mixing for 30 s to precipitate watersoluble proteins. Acetonitrile (1 mL) containing 400 ng/ml voriconazole as an internal standard was added to each plasma sample followed by vortex mixing for 1.5 min. The samples were then centrifuged at 3000×g for 15 min. The supernatants were then extracted and transferred to a clean centrifuge tube and seated in an aluminum heating block (70° C.) under a stream of nitrogen until dry. Samples were reconstituted with 250 μL mobile phase (62% acetonitrile: 38% 0.05 M potassium phosphate monobasic buffer adjusted to pH 6.7 with NaOH) and vortex mixed for 1 min. The samples were then centrifuged for an additional 15 min and subsequently a 150-μL aliquot of the supernatant was extracted and filled into low volume HPLC vial inserts. Each sample was analyzed using the Shimadzu VP-AT series LC10 HPLC system with a photodiode array detector, and extracting at a wavelength of 263 nm (λmax). An Altima® 5 μm C-18 HPLC column (250×4.6 mm) was used in the analysis. The column was maintained at a temperature of 37° C. for the duration of the injection set.

Non-compartmental analysis for extravascular input was performed on the data using WinNonlin version 4.1 (Pharsight Corporation, Mountain View, Calif.). By this method of analysis, Tmax and Cmax were determined directly from the empirical data, area under the plasma concentration—time curve (AUC) was calculated by the linear trapezoidal method, and t½ was determined by calculation of the lambda z parameter.

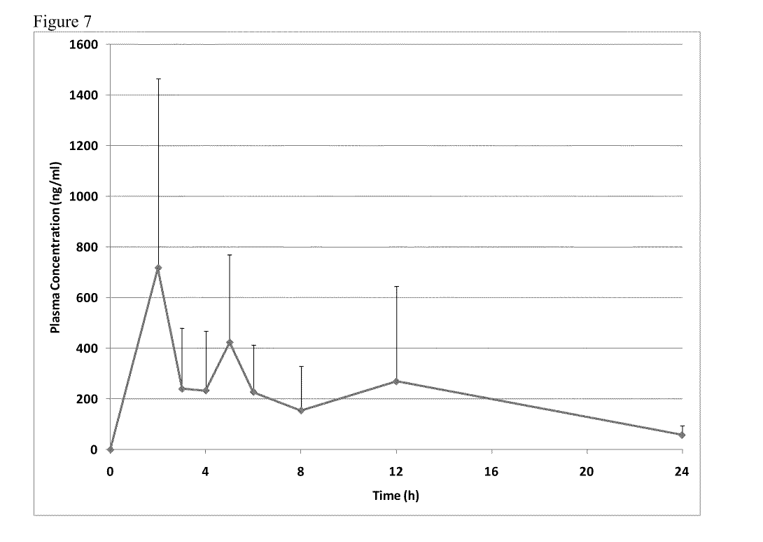

FIG. 7 shows the in-vivo animal model testing results for the poly(methacylic acid)-co-poly(methyl methacrylate) (PMAA-co-PMMA) coated amorphous Itraconazole (ITZ) particles prepared via a hot melt extrusion process. The results shown in FIG. 7 indicate that ITZ was present in the blood serum of the rats after dosing at ng/ml levels over a 24 hour period. After pharmacokinetic analysis the Cmax of this study was determined to be 717 (ng/ml), the Tmax was 2 hours, the AUC was 5278 (ng*hr/ml), and the T½ was 8.3 hours.

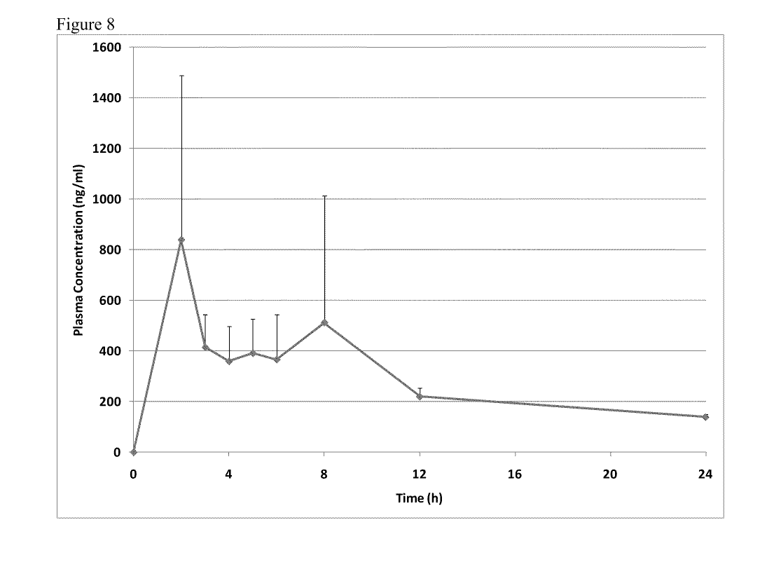

FIG. 8 shows the in-vivo animal model testing results for the perfluorohexane coated amorphous Itraconazole (ITZ) particles prepared via a hot melt extrusion process. The results shown in FIG. 8 indicate that ITZ was present in the blood serum of the rats after dosing at ng/ml levels over a 24 hour period. After pharmacokinetic analysis the Cmax of this study was determined to be 839 (ng/ml), the Tmax was 2 hours, the AUC was 7098 (ng*hr/ml), and the T½ was 10.8 hours.

### Example 6

### Amorphous Itraconazole Particles with PMAA-co-PMMA Coating (Antisolvent)

A plasma enhanced chemical vapor deposition process was employed using both methacrylic acid (MAA) and methyl methacrylate (MMA) monomers to deposit a thin film on the surface of an amorphous Itraconazole (ITZ) drug particle. The amorphous ITZ drug particles were prepared using an antisolvent precipitation process and were prepared from a pre-precipitation blend of crystalline ITZ, Polyvinyl pyrrole (PVP) K17, and Lecithin (1:1:0.25). The final potency of the as prepared amorphous antisolvent precipitation particles was 24.33% (SD of 0.58%) as measured by high pressure liquid chromatography. The film was deposited using a variable duty cycle and variable power 13.56 MHz plasma enhanced chemical vapor deposition coating process. During the first stage of the coating process a peak power of 15 watts and a duty cycle of 5 ms on and 30 ms off was utilized for 60 minutes at a constant reactor pressure of 100 mTorr. After 60 minutes the coating process was switched to a peak power of 22 watts and a duty cycle of 0.5 ms on and 30 ms off for an additional 60 minutes at a constant reactor pressure of 100 mTorr. During both stages of the coating process the monomer flow rates of methacrylic acid and methyl methacrylate were maintained independently at 75 and 25 sccm, respectively. This process yielded a stable and sufficiently thick gradient layered coating on the amorphous antisolvent precipitation particles. After coating the amorphous antisolvent precipitation particles were tested using differential scanning calorimetry and showed no recrystallization occurred during the coating process. The potency of the particles after coating was also tested and was measured to be 20.70% (SD of 0.22%).

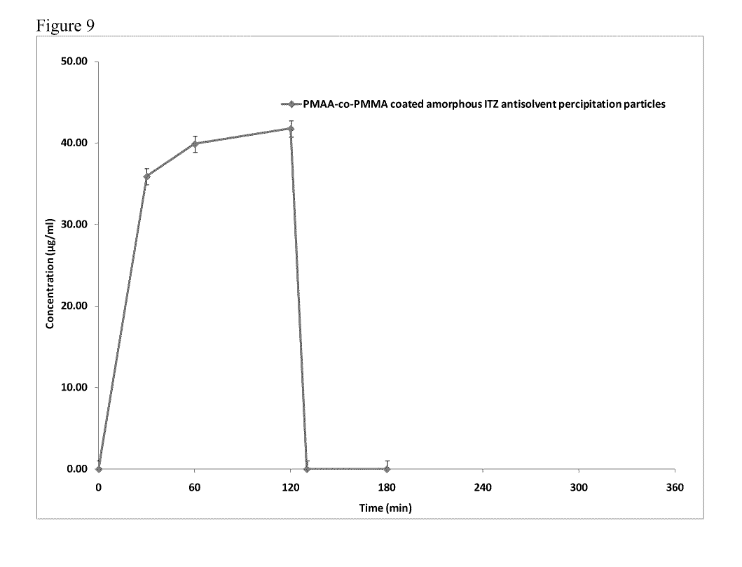

Supersaturation in the Stomach Environment FIG. 9 shows the dissolution testing results for the poly(methacylic acid)-co-poly(methyl methacrylate) (PMAA-co-PMMA) coated amorphous Itraconazole antisolvent precipitation particles. Dissolution testing was performed according to USP 29 Apparatus 2 guidelines (paddle method) at 50 rpm and a constant bath temperature of 37.0+/−0.2° C. The dissolution method utilized was in accordance with the USP 29 dissolution testing specifications for delayed-release dosage forms Method A. In this test 6 mg ITZ equivalent (based on the measured potency), which corresponds to 20 times the saturation solubility of ITZ in acidic media (4 μg/ml)1, was added to each dissolution vessel. Testing was conducted for 2 hrs in 75 ml of 0.1 N HCl followed by a pH-adjustment to 6.8+/−0.5 with the addition of 25 ml of tribasic sodium phosphate solution. The solubility of ITZ in neutral media is considerably lower at (˜1 ng/ml)1. The results shown in FIG. 9 indicate that dissolution and supersaturation of the poly(methacylic acid)-co-poly(methyl methacrylate) (PMAA-co-PMMA) coated amorphous Itraconazole antisolvent precipitation particles occurs nearly instantaneously in the acidic environment. The first measurement taken at the 30 minutes time point shows an ITZ concentration of 35.89 μg/ml that is approximately 10 times the equilibrium solubility of ITZ in acidic media. Additionally the supersaturation level continues to increase, until the final time point at 120 minutes before switching to a neutral pH, to a concentration of 41.76 μg/ml. After the addition of the tribasic sodium phosphate solution, which representative of the conditions of the upper small intestine, there is a rapid decline in the ITZ concentration until the measured level is below the detection limit of the instrument, at the 130 min time point, presumably due to the precipitation of the ITZ out of solution.

### Example 7

### Amorphous Itraconazole Particles with Perfluorohexane Coating (Antisolvent)

A plasma enhanced chemical vapor deposition process was employed using perfluorohexane (C6F14) monomer to deposit a thin film on the surface of an amorphous Itraconazole (ITZ) drug particle. The amorphous ITZ drug particles were prepared using an antisolvent precipitation process and were prepared from a pre-precipitation blend of crystalline ITZ, Polyvinyl pyrrole (PVP) K17, and Lecithin (1:1:0.25). The final potency of the as prepared amorphous antisolvent precipitation particles was 24.33% (SD of 0.58%) as measured by high pressure liquid chromatography. The coating was deposited using a 13.56 MHz plasma with a peak power of 150 watts and a duty cycle of 10 ms on and 40 ms off. The reaction chamber was maintained at a pressure of 160 mTorr with a monomer flow rate of 100 sccm for 90 minutes. This process yielded a stable and sufficiently thick gradient layered coating on the amorphous antisolvent precipitation particles. After coating the amorphous antisolvent precipitation particles were tested using differential scanning calorimetry and showed no recrystallization occurred during the coating process. The potency of the particles after coating was also tested and was measured to be 18.84% (SD of 1.28%).

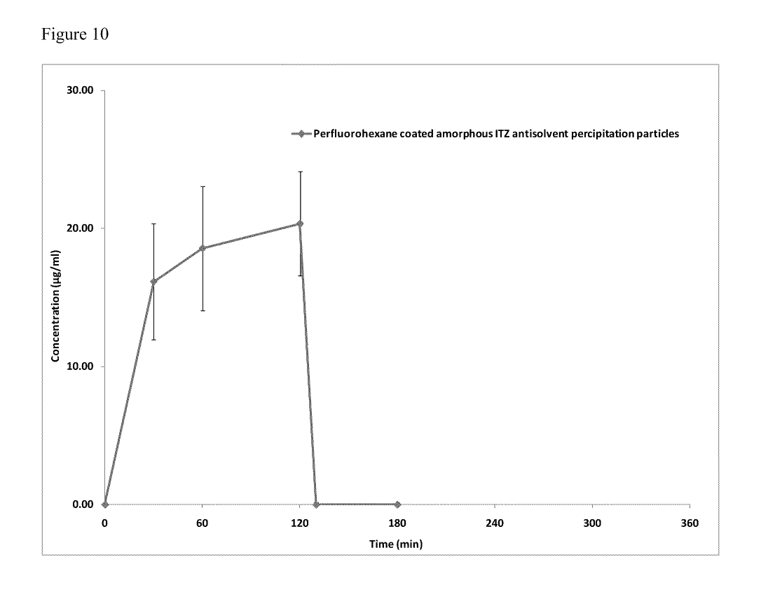

Supersaturation in the Stomach Environment. FIG. 10 shows the dissolution testing results for the perfluorohexane coated amorphous Itraconazole antisolvent precipitation particles. Dissolution testing was performed according to USP 29 Apparatus 2 guidelines (paddle method) at 50 rpm and a constant bath temperature of 37.0+/−0.2° C. The dissolution method utilized was in accordance with the USP 29 dissolution testing specifications for delayed-release dosage forms Method A. In this test 6 mg ITZ equivalent (based on the measured potency), which corresponds to 20 times the saturation solubility of ITZ in acidic media (4 μg/ml)1, was added to each dissolution vessel. Testing was conducted for 2 hrs in 75 ml of 0.1 N HCl followed by a pH-adjustment to 6.8+/−0.5 with the addition of 25 ml of tribasic sodium phosphate solution. The solubility of ITZ in neutral media is considerably lower at (˜1 ng/ml)1. The results shown in FIG. 10 indicate that dissolution and supersaturation of the perfluorohexane coated amorphous Itraconazole antisolvent precipitation particles occurs nearly instantaneously in the acidic environment. The first measurement taken at the 30 minute time point shows an ITZ concentration of 16.14 μg/ml which is approximately 4 times the equilibrium solubility of ITZ in acidic media. Additionally the supersaturation level continues to increase, until the final time point at 120 minutes before switching to a neutral pH, to a concentration of 20.33 μg/ml. After the addition of the tribasic sodium phosphate solution, which representative of the conditions of the upper small intestine, there is a rapid decline in the ITZ concentration until the measured level is below the detection limit of the instrument, at the 130 min time point, presumably due to the precipitation of the ITZ out of solution.

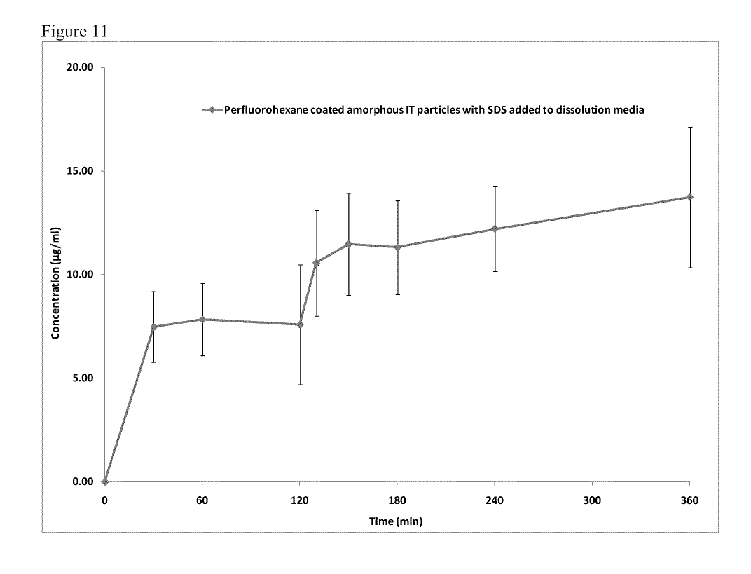

Supersaturation in the Stomach Environment (Surfactant Added). FIG. 11 shows the dissolution testing results for the perfluorohexane coated amorphous Itraconazole antisolvent precipitation particles with sodium dodecyl sulfate (SDS) surfactant added to the dissolution media. Dissolution testing was performed according to USP 29 Apparatus 2 guidelines (paddle method) at 50 rpm and a constant bath temperature of 37.0+/−0.2° C. The dissolution method utilized was in accordance with the USP 29 dissolution testing specifications for delayed-release dosage forms Method A. In this test 6 mg ITZ equivalent (based on the measured potency), which corresponds to 20 times the saturation solubility of ITZ in acidic media (4 μg/ml)1, was added to each dissolution vessel. Testing was conducted for 2 hrs in 75 ml of 0.1 N HCl followed by a pH-adjustment to 6.8+/−0.5 with the addition of 25 ml of tribasic sodium phosphate solution. The solubility of ITZ in neutral media is considerably lower at (˜1 ng/ml)1. Additionally, a small amount of SDS surfactant (conc. of 0.07%) was added to the dissolution media to help ensure wetting of the very hydrophobic perfluorohexane coated particles.

FIG. 11 indicate that dissolution and supersaturation of the perfluorohexane coated amorphous Itraconazole antisolvent precipitation particles occurs nearly instantaneously in the acidic environment. The first measurement taken at the 30 minute time point shows an ITZ concentration of 7.46 μg/ml which is approximately 2 times the equilibrium solubility of ITZ in acidic media. Additionally the supersaturation level continues to increase, until the final time point of the study at 360 minutes, to a concentration of 13.73 μg/ml.

Agents of the present invention may be Biopharmaceutics Classification System (BCS) Class II categorized compositions and water or moisture sensitive composition. The BCS Systems provides and important classification for the benefits of the inventions. The BCS Systems is as includes a high permeability and high solubility for BCS class I, high permeability and low solubility for BCS class II, low permeability and high solubility for BCS class III, and low permeability and low solubility for BCS class IV. A drug substance is considered HIGHLY SOLUBLE when the highest dose strength is soluble in <250 ml water over a pH range of 1 to 7.5. A drug substance is considered highly permeable when the extent of absorption in humans is determined to be >90% of an administered dose, based on mass-balance or in comparison to an intravenous reference dose. A drug product is considered to be rapidly dissolving when >85% of the labeled amount of drug substance dissolves within 30 minutes using USP apparatus 1 or II in a volume of <900 ml buffer solutions. Of course it should be recognized that the challenges and impact of solubility, permeability and delivery system transit time within entire GI tract becomes much more significant and complex with heterogeneity in pH, intestinal metabolism and permeability (e.g., Dressman et al. Pharm. Technology (2001); Vol. 25, No. 7, pp 68-76). A severe limitation in the oral bioavailability of class II compounds is that dissolution takes longer than the transit time through their absorptive sites, resulting in incomplete bioavailability (Dressman and Reppas, 2000).

The solubility class boundary is based on the highest dose strength of an immediate release (“IR”) formulation and a pH-solubility profile of the test drug in aqueous media with a pH range of 1 to 7.5. Solubility can be measured by the shake-flask or titration method or analysis by a validated stability-indicating assay. A drug substance is considered highly soluble when the highest dose strength is soluble in 250 ml or less of aqueous media over the pH range of 1-7.5. The volume estimate of 250 ml is derived from typical bioequivalence (BE) study protocols that prescribe administration of a drug product to fasting human volunteers with a glass (about 8 ounces) of water. In the absence of evidence suggesting instability in the gastrointestinal tract, a drug is considered highly soluble when 90% or more of an administered dose, based on a mass determination or in comparison to an intravenous reference dose, is dissolved.

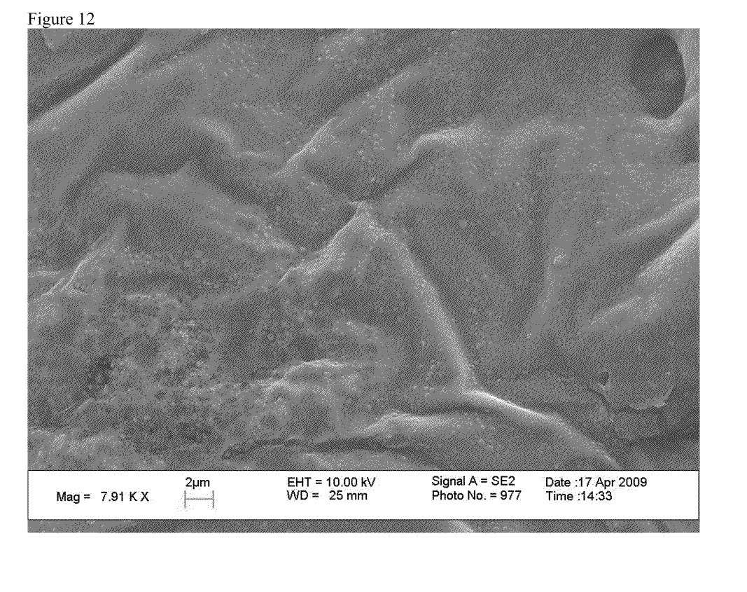

FIG. 12 depicts a SEM image showing the surface morphology of uncoated amorphous ITZ particles at a magnification of 7910×. Image was taken using a Leo 1530 scanning electron microscope.

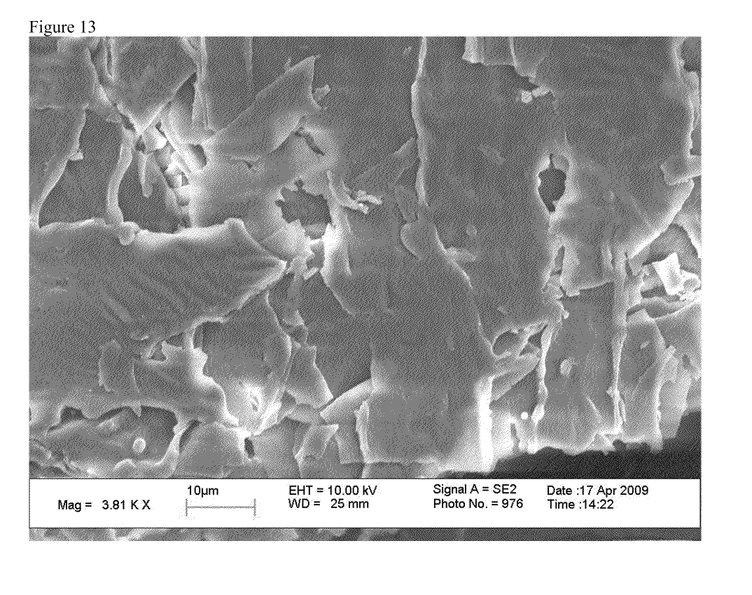

FIG. 13 depicts a SEM image showing the surface morphology of C6F14 coated amorphous ITZ particles at a magnification of 3810×. Image was taken using a Leo 1530 scanning electron microscope.

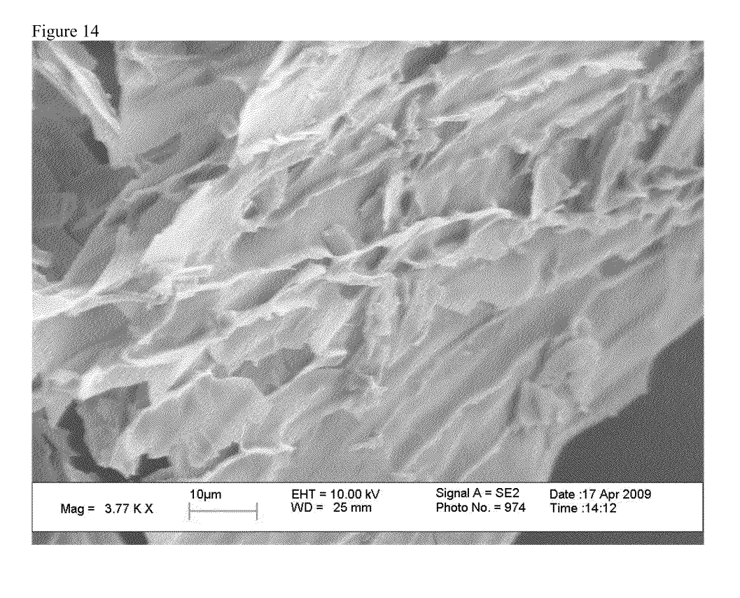

FIG. 14 depicts a SEM image showing the surface morphology of PMAA-co-PMMA coated amorphous ITZ particles at a magnification of 3770×. Image was taken using a Leo 1530 scanning electron microscope.

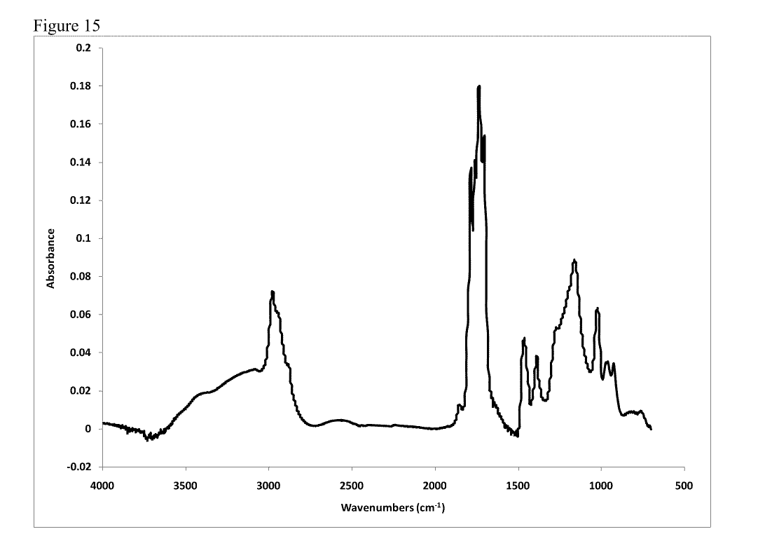

FIG. 15 depicts Fourier transform infrared spectroscopy analysis of the PMAA-co-PMMA coating deposited on a potassium bromide (KBr) powder using the plasma enhanced chemical vapor deposition technique.

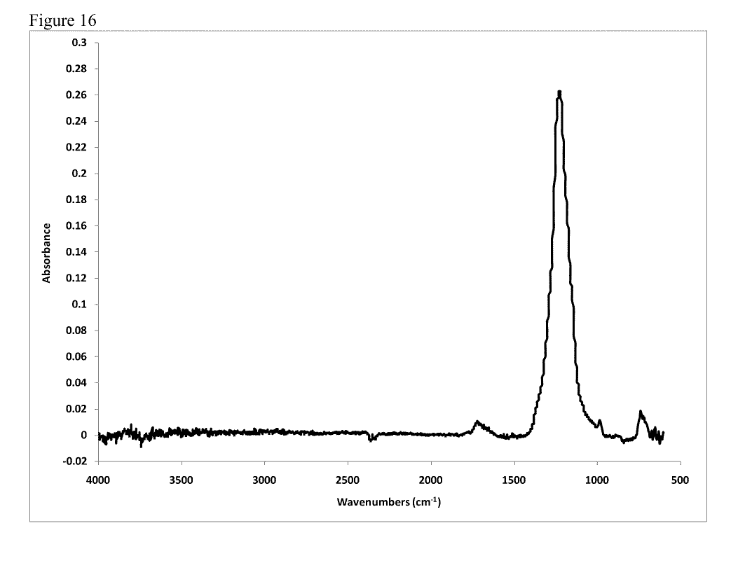

FIG. 16 depicts Fourier transform infrared spectroscopy analysis of the C6F14 coating deposited on a KBr powder using the plasma enhanced chemical vapor deposition technique.

Class II drugs are particularly insoluble, or slow to dissolve, but readily are absorbed from solution by the lining of the stomach and/or the intestine. Prolonged exposure to the lining of the GI tract is required to achieve absorption. Such drugs are found in many therapeutic classes. A class of particular interest is antifungal agents, such as itraconazole.

Based on the BCS, low-solubility compounds are compounds whose highest dose is not soluble in 250 mL or less of aqueous media from pH 1.2 to 7.5 at 37.degree. C. See Cynthia K. Brown, et al., “Acceptable Analytical Practices for Dissolution Testing of Poorly Soluble Compounds”, Pharmaceutical Technology (December 2004).

The permeability class boundary is based, directly, on measurements of the rate of mass transfer across human intestinal membrane, and, indirectly, on the extent of absorption (fraction of dose absorbed, not systemic bioavailability) of a drug substance in humans. The extent of absorption in humans is measured using mass-balance pharmacokinetic studies; absolute bioavailability studies; intestinal permeability methods; in vivo intestinal perfusion studies in humans; and in vivo or in situ intestinal perfusion studies in animals. In vitro permeation experiments can be conducted using excised human or animal intestinal tissue and in vitro permeation experiments can be conducted with epithelial cell monolayers. Alternatively, nonhuman systems capable of predicting the extent of drug absorption in humans can be used (e.g., in vitro epithelial cell culture methods). A drug substance is considered highly permeable when the extent of absorption in humans is determined to be greater than 90% of an administered dose, based on mass-balance or in comparison to an intravenous reference dose. A drug substance is considered to have low permeability when the extent of absorption in humans is determined to be less than 90% of an administered dose, based on mass-balance or in comparison to an intravenous reference dose. An IR drug product is considered rapidly dissolving when no less than 85% of the labelled amount of the drug substance dissolves within 30 minutes, using U.S. Pharmacopoeia (USP) Apparatus I at 100 rpm (or Apparatus II at 50 rpm) in a volume of 900 ml or less in each of the following media: (1) 0.1 N HCl or Simulated Gastric Fluid USP without enzymes; (2) a pH 4.5 buffer; and (3) a pH 6.8 buffer or Simulated Intestinal Fluid USP without enzymes.

Many of the known class II drugs are hydrophobic, and have historically been difficult to administer. Moreover, because of the hydrophobicity, there tends to be a significant variation in absorption depending on whether the patient is fed or fasted at the time of taking the drug. This in turn can affect the peak level of serum concentration, making calculation of dosage and dosing regimens more complex. Many of these drugs are also relatively inexpensive, so that simple formulation methods are required and some inefficiency in yield is acceptable.

In the preferred embodiment the drug is itraconazole or a related drug, such as fluconazole, terconazole, ketoconazole, and saperconazole. Itraconazole is a class II medicine used to treat fungal infections and is effective against a broad spectrum of fungi including dermatophytes (tinea infections), candida, malassezia, and chromoblastomycosis. Itraconazole works by destroying the cell wall and critical enzymes of yeast and other fungal infectious agents. Itraconazole can also decrease testosterone levels, which makes it useful in treating prostate cancer and can reduce the production of excessive adrenal corticosteroid hormones, which makes it useful for Cushing's syndrome. Itraconazole is available in capsule and oral solution form. For fungal infections the recommended dosage of oral capsules is 200-400 mg once a day.

Itraconazole has been available in capsule form since 1992, in oral solution form since 1997, and in an intravenous formulation since 1999. Since Itraconazole is a highly lipophilic compound, it achieves high concentrations in fatty tissues and purulent exudates. However, its penetration into aqueous fluids is very limited. Gastric acidity and food heavily influence the absorption of the oral formulation (Bailey, et al., Pharmacotherapy, 10: 146-153 (1990)). The absorption of itraconazole oral capsule is variable and unpredictable, despite having a bioavailability of 55%.

Other suitable drugs include class II anti-infective drugs, such as griseofulvin and related compounds such as griseoverdin; some anti malaria drugs (e.g. Atovaquone); immune system modulators (e.g cyclosporine); and cardiovascular drugs (e.g. digoxin and spironolactone); and ibuprofen. In addition, sterols or steroids may be used. Drugs such as Danazol, carbamazepine, and acyclovir may also be used in the compositions.

Danazol is derived from ethisterone and is a synthetic steroid. Danazol is designated as 17a-Pregna-2,4-dien-20-yno[2,3-d]isoxazol-17-ol, has the formula of C.sub.22H.sub.27NO.sub.2, and a molecular weight of 337.46. Danazol is a synthetic steroid hormone resembling a group of natural hormones (androgens) that are found in the body. Danazol is used in the treatment of endometriosis. It is also useful in the treatment of fibrocystic breast disease and hereditary angioedema. Danazol works to reduce oestrogen levels by inhibiting the production of hormones called gonadotrophins by the pituitary gland. Gonadotrophins normally stimulate the production of sex hormones such as oestrogen and progestogen, which are responsible for body processes such as menstruation and ovulation.

Danazol is administered orally, has a bioavailability that is not directly dose-related, and a half-life of 4-5 hours. Dosage increases in danazol are not proportional to increases in plasma concentrations. It has been shown that doubling the dose may yield only a 30-40% increase in plasma concentration. Danazol peak concentrations occur within 2 hours, but the therapeutic effect usually does not occur for approximately 6-8 weeks after taking daily doses.

Acyclovir is a synthetic nucleoside analogue that acts as an antiviral agent. Acyclovir is available for oral administration in capsule, tablet, and suspension forms. It is a white, crystalline powder designated as 2-amino-1,9-dihydro-9-[(2-hydroxyethoxy)methyl]-6H-purin-6-one, has an empirical formula of C.sub.8H.sub.11N.sub.5O.sub.3 and a molecular weight of 225.

Acyclovir has an absolute bioavailability of 20% at a 200 mg dose given every 4 hours, with a half-life of 2.5 to 3.3 hours. In addition, the bioavailability decreases with increasing doses. Despite its low bioavailability, acyclovir is highly specific in its inhibitory activity of viruses due to its high affinity for thymidine kinase (TK) (encoded by the virus). TK converts acyclovir into a nucleotide analogue which prevents replication of viral DNA by inhibition and/or inactivation of the viral DNA polymerase, and through termination of the growing viral DNA chain.

Carbamazepine is used in the treatment of psychomotor epilepsy, and as an adjunct in the treatment of partial epilepsies. It can also relieve or diminish pain that is associated with trigeminal neuralgia. Carbamazepine given as a monotherapy or in combination with lithium or neuroleptics has also been found useful in the treatment of acute mania and the prophylactic treatment of bipolar disorders.

Carbamazepine is a white to off-white powder, is designated as 5H-dibenz[b,f]azepine-5-carboxamide, and has a molecular weight of 236.77. It is practically insoluble in water and soluble in alcohol and acetone. The absorption of carbamazepine is relatively slow, despite a bioavailability of 89% for the tablet form. When taken in a single oral dose, the carbamazepine tablets and chewable tablets yield peak plasma concentrations of unchanged carbamazepine within 4 to 24 hours. The therapeutic range for the steady-state plasma concentration of carbamazepine generally lies between 4 and 10 mcg/mL.

“Class II” drugs of the BCS system dissolve poorly in the gastrointestinal (GI) tract, but are readily absorbed from solution. Such drugs tend to show a significant difference in their eventual absorption, depending on whether the patient is recently fed versus fasting when taking an oral dose. These drugs may also pass through the GI tract with variable proportions of absorption. These effects make oral formulations of Class II drugs both important and difficult.

Three of the parameters that can be manipulated to improve the bioavailability of Class II drugs are (1) particle size, (2) particle dispersion, and (3) release rate. A variety of methods are available for providing drugs in a form which has a large surface, especially as small particles of a few microns in diameter or smaller. Besides fine grinding of crystals, the formation of microparticles from solution by precipitation, spray drying, freeze-drying, and similar methods is known. In addition, the drug solution can be coated onto small particles to achieve its dispersion, as described, for example, in U.S. Pat. No. 5,633,015 to Gilis et al.

Micronized drug on its own tends to re-agglomerate when administered, and this decreases the advantage of improved release kinetics obtained by micronization. Hence, it is also necessary to prevent fine particles of drug from aggregating in formulation. Polymers and other excipients may form a matrix that separates the micronized particles as they are released. Generally, hydrophilic materials, whether polymers or small molecules, are mixed with the fine particles either during or after manufacture. The dried composite materials are typically tableted or put in a capsule. Then, when the capsule or tablet enters the stomach or intestine, the finely dispersed drug is dispersed into the gastrointestinal fluid without aggregating. Such compositions are sometimes referred to as “immediate release”.

Immediate release solid oral dosage forms are typically prepared by blending drug particles with fillers, such as lactose and microcrystalline cellulose; glidants, such as talc and silicon dioxide; disintegrants, such as starch, crosprovidone; and/or lubricants, such as magnesium stearate; and compressing the mixture into the form of a tablet. Alternately the mixture may be filled into a standard capsule, providing a simple oral dosage form.

Hydrophilic polymers may also be used to form a matrix with hydrophobic drugs to separate drug particles, improve wetting and improve dissolution. Polymers such as hydroxyl propylcellulose (HPC), hydroxypropylmethylcellulose (HPMC), and carboxymethylcellulose (CMC) are commonly used for this purpose. The matrix may be formed by blending and direct compression, hot melt extrusion, spray-drying, spray-congealing, wet granulation and extrusion-spheronization.

Although these techniques are effective, in the abstract, the rate of absorption is dependant on whether or not the patient ate when taking the drug. For example, the absorption of the drug is significantly higher when the drug is taken with a meal than when it is not. This may be due to competition between dissolution of drug, and aggregation of drug particles as the water-soluble material dissolves. The latter effect may be minimized in the presence of food.

The invention may be used for a wide range of low aqueous solubility and dissolution rate active agents or bioactive compounds of the group of ACE inhibitors, adenohypophoseal hormones, adrenergic neuron blocking agents, adrenocortical steroids, inhibitors of the biosynthesis of adrenocortical steroids, alpha-adrenergic agonists, alpha-adrenergic antagonists, selective alpha.sub.2-adrenergic agonists, analgesics, antipyretics and anti-inflammatory agents, androgens, anesthetics, antiaddictive agents, antiandrogens, antiarrhythmic agents, antiasthmatic agents, anticholinergic agents, anticholinesterase agents, anticoagulants, antidiabetic agents, antidiarrheal agents, antidiuretics, antiemetic and prokinetic agents, antiepileptic agents, antiestrogens, antifungal agents, antihypertensive agents, antimicrobial agents, antimigraine agents, antimuscarinic agents, antineoplastic agents, antiparasitic agents, antiparkinsons agents, antiplatelet agents, antiprogestins, antithyroid agents, antitussives, antiviral agents, a typical antidepressants, azaspirodecanediones, barbituates, benzodiazepines, benzothiadiazides, beta-adrenergic agonists, beta-adrenergic antagonists, selective beta.sub.1-adrenergic antagonists, selective beta.sub.2-adrenergic agonists, bile salts, agents affecting volume and composition of body fluids, butyrophenones, agents affecting calcification, calcium channel blockers, cardiovascular drugs, catecholamines and sympathomimetic drugs, cholinergic agonists, cholinesterase reactivators, dermatological agents, diphenylbutylpiperidines, diuretics, ergot alkaloids, estrogens, ganglionic blocking agents, ganglionic stimulating agents, hydantoins, agents for control of gastric acidity and treatment of peptic ulcers, haematopoietic agents, histamines, histamine antagonists, 5-hydroxytryptamine antagonists, drugs for the treatment of hyperlipoproteinemia, hypnotics and sedatives, immunosuppressive agents, laxatives, methylxanthines, monoamine oxidase inhibitors, neuromuscular blocking agents, organic nitrates, opiod analgesics and antagonists, pancreatic enzymes, phenothiazines, progestins, prostaglandins, agents for the treatment of psychiatric disorders, retinoids, sodium channel blockers, agents for spasticity and acute muscle spasms, succinimides, thioxanthines, thrombolytic agents, thyroid agents, tricyclic antidepressants, inhibitors of tubular transport of organic compounds, drugs affecting uterine motility, vasodilators, vitamins and the like, alone or in combination. Although extensive, this list is not intended to be comprehensive.

In another embodiment the dosage form of present invention is used for the poorly soluble drug is selected from the group consisting of carbamazepine, dapsone, griseofulvin, indinavir, nifedipine, nitrofurantion, phentytoin, ritonavir, saquinavir, sulfamethoxazole, valproic acid and trimethoprin.

In yet an other embodiment of present invention the dosage form comprises a drug selected from the group of compounds consisting of albendazole, amitryptyline, artemether, lumefantrine, chloropromazine, ciprofloxac in, clofazimine, efavirenz, lopinavir, folic acid, glibenclamide, haloperidol, ivermectin, mebendazole, niclosamide, pyrantel, pyrimethamine, retinol vitamin, sulfadiazine, sulfasalazine, triclabendazole.

The present invention shows that deposition of a polymer film or coating layer using plasma polymerization is a new and improved way to introduce and control the release of an agent into an environment. Using systems, methods, and compositions of the present invention, one can prepare any encapsulated particle coated with any degradable, nondegradable, and/or solubility enhancing polymer and alter particle release rates to control particle introduction into an environment and even to achieve supersaturation. Supersaturation in the gastrointestinal tract may be altered by composition of the coating layers as well as release in the environment. The control of particle introduction into the environment may be a temporal and/or site-specific control. For example, polymer film deposition may be controlled by altering reaction conditions, such as power input, peak power, coating time, duty cycle, flow rate of the carbonaceous compound, reactor pressure, and/or quantity of particles during preparation of the coated particles. These conditions control aspects of the coating or polymer film, including polymer film growth, film thickness, the density of polar groups, the number of functional groups, the hydrophilicity or hydrophobicity, molecular weight, wettability, linearity, and extent of cross-linkages in the polymer. In this way, a polymer film of the present invention may be finely tuned in order to obtain any required combination of temporal and/or site-specific release of particles into an environment.

The present invention also provides for compositions prepared by systems and methods described herein. Such compositions, systems and/or methods disclosed and claimed herein can be made and executed without undue experimentation in light may include one or more agents as well as one or more different types of particles. Indeed, such variations in the present invention may be specifically manufactured to optimally control release of one or more particles into an environment. Optimally control may include combining particles with similar or different coatings wherein the differences include coating composition, thickness, number and/or type of functional group, hydrophobicity, hydrophilicity, wettability, linearity, cross-linking, and combinations thereof. With the present invention, one or more different compositions may also be combined to yield a desired particle release property.

While specific alternatives to steps of the compositions and methods of this invention have been described in terms of preferred embodiments herein, additional alternatives not specifically disclosed but known in the art are intended to fall within the scope of the invention. Thus, it is understood that other applications of the present invention will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within upon reading the spirit, scope described embodiment and concept after consideration of the invention as defined by the appended claims and drawings.

What is claimed is:

1) A system for delivery of an enteric coated active agent generally resistant to disintegration in an neutral environment comprising
one or more active agents encapsulated by a polymer coating formed by chemical vapor deposition of one or more monomers on the one or more active agents to form a chemical vapor deposition polymer coating that controls the release of the one or more active agents in the gastrointestinal tract.

2) The method of claim 1, wherein the one or more active agents comprise analgesic agents, anti-inflammatory agents, anti-infective agents, proteinaceous agents, enzymatic agents, or a combination thereof.

3) The system of claim 1, wherein the one or more active agents comprise Itraconazole, aspirin, Ketoprofen, Albuterol sulfate, cabamazepine, cyclosporin A (CsA), Danazol, ketoconazole, Itraconazole, voriconazole, Naproxen, Repaglinide, Tacrolimus, bovine insulin, Beclomethasone, Buprenorphine, Methadone, Atovaquone, Ranolazine or combinations thereof.

4) The system of claim 1, wherein the one or more active agents are coated with at least two layers of the high surface coverage chemical vapor deposition polymer coat.

5) The system of claim 1, wherein the high surface coverage chemical vapor deposition polymer coat comprises two or more layers of different coatings.

6) The system of claim 6, wherein the two or more layers of different coatings are of different thicknesses.

7) The system of claim 1, wherein the one or more monomers comprise ethylene, vinyl alcohol, acrylic acid, carbophil, ethylene glycol, glycolic acid, saccharide, lactic acid, esters, ortho esters, phosphazenes, anhydrides, amides, perfluoroalkenes or a combination thereof.

8) The system of claim 1, wherein the one or more monomers comprise hexamethyldisiloxane, perfluorohexane, methacrylic monomers selected from methacrylic acid (MAA), methyl methacrylate (MMA), poly(methacylic acid)-co-poly(methyl methacrylate) (PMAA-co-PMMA), 2,3,5-trimethyl-3-hexene, 2,3,5-trimethyl-2-hexene, 2,4,5-trimethyl-2-hexene, perfluoroalkane monomers selected from CnF(2n+2) monomers like C2F6, C3F8, C4F10, C5F12, C6F14, C9F18, C6F12, C7F14, and C8F16 or combinations thereof.

9) The system of claim 1, wherein the surface coverage chemical vapor deposition polymer coat thickness increases the force of adhesion between the higher surface coverage chemical vapor deposition polymer coat and the one or more active agents that leads to a faster rate of removal of the coating upon submersion in an aqueous medium and hence a faster dissolution rate.

10) A method of encapsulating one or more active agents in a chemical vapor deposition layer for controlled release in the intestinal tract comprising the steps of:
providing one or more active agents in a reaction chamber;
adding one or more monomers to the reaction chamber;
forming a plasma of the one or more monomers to make a chemical vapor;
depositing the chemical vapor on the one or more active agents to encapsulate the one or more active agents in a chemical vapor deposition polymer coating for controlled release in the intestinal tract.

11) The method of claim 10, wherein the one or more active agents comprise one or more Biopharmaceutics Classification System (BCS) Class II compositions.

12) The method of claim 10, wherein the one or more active agents comprise analgesic, anti-inflammatory agents, anti-infectives, proteinaceous agents, enzymatic agents, or a combination thereof.

13) The method of claim 10, wherein the one or more active agents comprise Itraconazole, aspirin, Ketoprofen, Albuterol sulfate, cabamazepine, cyclosporin A (CsA), Danazol, ketoconazole, Itraconazole, voriconazole, Naproxen, Repaglinide, Tacrolimus, bovine insulin, Beclomethasone, Buprenorphine, Methadone, Atovaquone, Ranolazine or combinations thereof.

14) The method of claim 10, wherein the one or more active agents are coated with at least two layers of the high surface coverage chemical vapor deposition polymer coat.

15) The method of claim 10, wherein the high surface coverage chemical vapor deposition polymer coat comprises two or more layers of different coatings.

16) The method of claim 15, wherein the two or more layers of different coatings are of different thicknesses.

17) The method of claim 10, wherein the one or more monomers comprise ethylene, vinyl alcohol, acrylic acid, carbophil, ethylene glycol, glycolic acid, saccharide, lactic acid, esters, ortho esters, phosphazenes, anhydrides, amides, perfluoroalkenes or a combination thereof.

18) The method of claim 10, wherein the one or more monomers comprise hexamethyldisiloxane, perfluorohexane, methacrylic monomers selected from methacrylic acid (MAA), methyl methacrylate (MMA), poly(methacylic acid)-co-poly(methyl methacrylate) (PMAA-co-PMMA), 2,3,5-trimethyl-3-hexene, 2,3,5-trimethyl-2-hexene, 2,4,5-trimethyl-2-hexene, perfluoroalkane monomers selected from CnF(2n+2) monomers like C2F6, C3F8, C4F10, C5F12, C6F14, C9F18, C6F12, C7F14, and C8F16 or combinations thereof.

19) The method of claim 10, wherein the surface coverage chemical vapor deposition polymer coat thickness increases the force of adhesion between the higher surface coverage chemical vapor deposition polymer coat and the one or more active agents that leads to a faster rate of removal of the coating upon submersion in an aqueous medium and hence a faster dissolution rate.

20) A method for delivering one or more active agents to achieve supersaturation in the gastrointestinal tract comprising the steps of:
providing a pharmaceutical composition having a chemical vapor deposition layer to control the release to a subject, wherein the pharmaceutical composition comprises one or more active agents encapsulated with a polymer coating that has been deposited by chemical vapor deposition for controlled release at a pH 4.5 or higher in the intestinal tract.

21) An enteric coated pharmaceutical composition having a chemical vapor deposition layer comprising:
one or more active agents encapsulated by a polymer coating formed by chemical vapor deposition on the one or more active agents to form a chemical vapor deposition polymer coating that controls the release of the one or more active agents at a pH 4.5 or higher.

22) The composition of claim 21, wherein the one or more active agents comprise one or more Biopharmaceutics Classification System (BCS) Class II compositions, one or more moisture sensitive compositions, analgesic agents, anti-inflammatory agents, anti-infective agents, proteinaceous agents, enzymatic agents, or a combination thereof.

23) The composition of claim 21, wherein the enteric coated pharmaceutical composition is impervious to moisture and forms a moisture barrier.

24) The composition of claim 21, wherein the one or more active agents comprise Itraconazole, aspirin, Ketoprofen, Albuterol sulfate, cabamazepine, cyclosporin A (CsA), Danazol, ketoconazole, Itraconazole, voriconazole, Naproxen, Repaglinide, Tacrolimus, bovine insulin, Beclomethasone, Buprenorphine, Methadone, Atovaquone, Ranolazine or combinations thereof.

25) The composition of claim 21, wherein the one or more active agents are coated with at least two layers of the polymer coating.

26) The composition of claim 21, wherein the chemical vapor deposition polymer coating are two or more layers of different thicknesses.

27) The composition of claim 21, wherein the one or more monomers comprise ethylene, vinyl alcohol, acrylic acid, carbophil, ethylene glycol, glycolic acid, saccharide, lactic acid, esters, ortho esters, phosphazenes, anhydrides, amides, perfluoroalkenes or a combination thereof.

28) The composition of claim 21, wherein the one or more monomers comprise hexamethyldisiloxane, perfluorohexane, methacrylic monomers selected from methacrylic acid (MAA), methyl methacrylate (MMA), poly(methacylic acid)-co-poly(methyl methacrylate) (PMAA-co-PMMA), 2,3,5-trimethyl-3-hexene, 2,3,5-trimethyl-2-hexene, 2,4,5-trimethyl-2-hexene, perfluoroalkane monomers selected from CnF(2n+2) monomers like C2F6, C3F8, C4F10, C5F12, C6F14, C9F18, C6F12, C7F14, and C8F16 or combinations thereof.
